SEQUENCE LISTING (24 sequences)

SEQ ID NO: 1 - protein, 258 aa, synthetic construct
MTSSHAMNITPLAQLALLFSTLLLPGTQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGSHTVEENPSLFALEAVLIPVGTMGLIITLIFVYCWLERMPPIPPIKNLEDLVTEYQGNFSAWSGVSKGLTESLQPDYSERFCHVSEIPPKGGALGEGPGGSPCSLHSPYWPPPCYSLKPEA

SEQ ID NO: 2 - protein, 245 aa, synthetic construct
MTSSHAMNITPLAQLALLFSTLLLPGTQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGEAVLIPVGTMGLIITLIFVYCWLERMPPIPPIKNLEDLVTEYQGNFSAWSGVSKGLTESLQPDYSERFCHVSEIPPKGGALGEGPGGSPCSLHSPYWPPPCYSLKPEA

SEQ ID NO: 3 - protein, 420 aa, synthetic construct
MTSSHAMNITPLAQLALLFSTLLLPGTQAEMWHEGLEEASRLYFGERNVKGMFEVLEPLHAMMERGPQTLKETSFNQAYGRDLMEAQEWCRKYMKSGNVKDLTQAWDLYYHVFRRISKGKEILPMSWLRYLLLVLGCFSGFFSCVYILVKCRYLGPWLKTVLKCHIPDPSEFFSQLSSQHGGDLQKWLSSPVPLSFFSPSGPAPEISPLEVLDGDSKAVQLLLLQKDSAPLPSPSGHSQASCFTNQGYFFFHLPNALEIESCQVYFTYDPCVEEEVEEDGSRLPEGSPHPPLLPLAGEQDDYCAFPPRDDLLLFSPSLSTPNTAYGGSRAPEERSPLSLHEGLPSLASRDLMGLQRPLERMPEGDGEGLSANSSGEQASVPEGNLHGQDQDRGQGPILTLNTDAYLSLQELQAQDSVHLI

SEQ ID NO: 4 - protein, 440 aa, synthetic construct
MLLLVTSLLLCELPHPAFLLIPGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGSNTSKENPFLFALEAVVISVGSMGLIISLLCVYFWLYRAHFGTDETILDGKEYDIYVSYARNAEEEEFVLLTLRGVLENEFGYKLCIFDRDSLPGGIVTDETLSFIQKSRRLLVVLSPNYVLQGTQALLELKAGLENMASRGNINVILVQYKAVKETKVKELKRAKTVLTVIKWKGEKSKYPQGRFWKQLQVAMPVKKSPRRSSSDEQGLSYSSLKNVXXXXXXERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 5 - protein, 639 aa, synthetic construct
MLLLVTSLLLCELPHPAFLLIPEMWHEGLEEASRLYFGERNVKGMFEVLEPLHAMMERGPQTLKETSFNQAYGRDLMEAQEWCRKYMKSGNVKDLTQAWDLYYHVFRRISKGKDTIPWLGHLLVGLSGAFGFIILVYLLILKMFWIEATLLWRDIAKPYKTRNDGKLYDAYVVYPRNYKSSTDGASRVEHFVHQILPDVLENKCGYTLCIYGRDMLPGEDVVTAVETNIRKSRRHIFILTPQITHNKEFAYEQEVALHCALIQNDAKVILIEMEALSELDMLQAEALQDSLQHLMKVQGTIKWREDHIANKRSLNSKFWKHVRYQMPVPSKIPRKASSLTPLAAQKQXXXXXXNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 6 - protein, 465 aa, synthetic construct
MLLLVTSLLLCELPHPAFLLIPGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGSNTSKENPFLFALEAVVISVGSMGLIISLLCVYFWLLKMFWIEATLLWRDIAKPYKTRNDGKLYDAYVVYPRNYKSSTDGASRVEHFVHQILPDVLENKCGYTLCIYGRDMLPGEDVVTAVETNIRKSRRHIFILTPQITHNKEFAYEQEVALHCALIQNDAKVILIEMEALSELDMLQAEALQDSLQHLMKVQGTIKWREDHIANKRSLNSKFWKHVRYQMPVPSKIPRKASSLTPLAAQKQXXXXXXERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 7 - protein, 614 aa, synthetic construct
MLLLVTSLLLCELPHPAFLLIPEMWHEGLEEASRLYFGERNVKGMFEVLEPLHAMMERGPQTLKETSFNQAYGRDLMEAQEWCRKYMKSGNVKDLTQAWDLYYHVFRRISKGKDTIPWLGHLLVGLSGAFGFIILVYLLIYRAHFGTDETILDGKEYDIYVSYARNAEEEEFVLLTLRGVLENEFGYKLCIFDRDSLPGGIVTDETLSFIQKSRRLLVVLSPNYVLQGTQALLELKAGLENMASRGNINVILVQYKAVKETKVKELKRAKTVLTVIKWKGEKSKYPQGRFWKQLQVAMPVKKSPRRSSSDEQGLSYSSLKNVXXXXXXNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 8 - protein, 443 aa, synthetic construct
MLLLVTSLLLCELPHPAFLLIPGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEQKVPAPRYTVELACGFGATVLLVVILIVVYHVYWLEMVLFYRAHFGTDETILDGKEYDIYVSYARNAEEEEFVLLTLRGVLENEFGYKLCIFDRDSLPGGIVTDETLSFIQKSRRLLVVLSPNYVLQGTQALLELKAGLENMASRGNINVILVQYKAVKETKVKELKRAKTVLTVIKWKGEKSKYPQGRFWKQLQVAMPVKKSPRRSSSDEQGLSYSSLKNVXXXXXXERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 9 - protein, 641 aa, synthetic construct
MLLLVTSLLLCELPHPAFLLIPEMWHEGLEEASRLYFGERNVKGMFEVLEPLHAMMERGPQTLKETSFNQAYGRDLMEAQEWCRKYMKSGNVKDLTQAWDLYYHVFRRISKSRKNPIDHHSIYCIIAVCSVFLMLINVLVIILKMFWIEATLLWRDIAKPYKTRNDGKLYDAYVVYPRNYKSSTDGASRVEHFVHQILPDVLENKCGYTLCIYGRDMLPGEDVVTAVETNIRKSRRHIFILTPQITHNKEFAYEQEVALHCALIQNDAKVILIEMEALSELDMLQAEALQDSLQHLMKVQGTIKWREDHIANKRSLNSKFWKHVRYQMPVPSKIPRKASSLTPLAAQKQXXXXXXNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 10 - protein, 459 aa, synthetic construct
MLLLVTSLLLCELPHPAFLLIPGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESRKNPIDHHSIYCIIAVCSVFLMLINVLVIILKMFWIEATLLWRDIAKPYKTRNDGKLYDAYVVYPRNYKSSTDGASRVEHFVHQILPDVLENKCGYTLCIYGRDMLPGEDVVTAVETNIRKSRRHIFILTPQITHNKEFAYEQEVALHCALIQNDAKVILIEMEALSELDMLQAEALQDSLQHLMKVQGTIKWREDHIANKRSLNSKFWKHVRYQMPVPSKIPRKASSLTPLAAQKQXXXXXXERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 11 - protein, 625 aa, synthetic construct
MLLLVTSLLLCELPHPAFLLIPEMWHEGLEEASRLYFGERNVKGMFEVLEPLHAMMERGPQTLKETSFNQAYGRDLMEAQEWCRKYMKSGNVKDLTQAWDLYYHVFRRISKQKVPAPRYTVELACGFGATVLLVVILIVVYHVYWLEMVLFYRAHFGTDETILDGKEYDIYVSYARNAEEEEFVLLTLRGVLENEFGYKLCIFDRDSLPGGIVTDETLSFIQKSRRLLVVLSPNYVLQGTQALLELKAGLENMASRGNINVILVQYKAVKETKVKELKRAKTVLTVIKWKGEKSKYPQGRFWKQLQVAMPVKKSPRRSSSDEQGLSYSSLKNVXXXXXXNCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 12 - protein, 249 aa, synthetic construct
MLLLVTSLLLCELPHPAFLLIPGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGGGSGGGGSGGGGSWMVAVILMASVFMVCLALLGCFALLWCVYKKTKYAFSPRNSLPQHLKEFLGHPHHNTLLFFSFPLSDENDVFDKLSVIAEDSESGKQNPGDSCSLGTPPGQGPQS

SEQ ID NO: 13 - protein, 469 aa, synthetic construct
MLLLVTSLLLCELPHPAFLLIPEMWHEGLEEASRLYFGERNVKGMFEVLEPLHAMMERGPQTLKETSFNQAYGRDLMEAQEWCRKYMKSGNVKDLTQAWDLYYHVFRRISKGGGGSGGGGSGGGGSVIIFFAFVLLLSGALAYCLALQLYVRRRKKLPSVLLFKKPSPFIFISQRPSPETQDTIHPLDEEAFLKVSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 14 - protein, 243 aa, synthetic construct
MLLLVTSLLLCELPHPAFLLIPGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETTHDETVPSWMVAVILMASVFMVCLALLGCFALLWCVYKKTKYAFSPRNSLPQHLKEFLGHPHHNTLLFFSFPLSDENDVFDKLSVIAEDSESGKQNPGDSCSLGTPPGQGPQS

SEQ ID NO: 15 - protein, 464 aa, synthetic construct
MLLLVTSLLLCELPHPAFLLIPEMWHEGLEEASRLYFGERNVKGMFEVLEPLHAMMERGPQTLKETSFNQAYGRDLMEAQEWCRKYMKSGNVKDLTQAWDLYYHVFRRISKLTRQYFTVTNVIIFFAFVLLLSGALAYCLALQLYVRRRKKLPSVLLFKKPSPFIFISQRPSPETQDTIHPLDEEAFLKVSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 16 - protein, 356 aa, synthetic construct
MLLLVTSLLLCELPHPAFLLIPYPYDVPDYAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGGGSGGGGSGGGGSVLLVVILIVVYHVYWLEMVLFYRAHFGTDETILDGKEYDIYVSYARNAEEEEFVLLTLRGVLENEFGYKLCIFDRDSLPGGIVTDETLSFIQKSRRLLVVLSPNYVLQGTQALLELKAGLENMASRGNINVILVQYKAVKETKVKELKRAKTVLTVIKWKGEKSKYPQGRFWKQLQVAMPVKKSPRRSSSDEQGLSYSSLKNV

SEQ ID NO: 17 - protein, 354 aa, synthetic construct
MLLLVTSLLLCELPHPAFLLIPEMWHEGLEEASRLYFGERNVKGMFEVLEPLHAMMERGPQTLKETSFNQAYGRDLMEAQEWCRKYMKSGNVKDLTQAWDLYYHVFRRISKGGGGSGGGGSGGGGSIYCIIAVCSVFLMLINVLVIILKMFWIEATLLWRDIAKPYKTRNDGKLYDAYVVYPRNYKSSTDGASRVEHFVHQILPDVLENKCGYTLCIYGRDMLPGEDVVTAVETNIRKSRRHIFILTPQITHNKEFAYEQEVALHCALIQNDAKVILIEMEALSELDMLQAEALQDSLQHLMKVQGTIKWREDHIANKRSLNSKFWKHVRYQMPVPSKIPRKASSLTPLAAQKQ

SEQ ID NO: 18 - protein, 351 aa, synthetic construct
MLLLVTSLLLCELPHPAFLLIPGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEQKVPAPRYTVELACGFGATVLLVVILIVVYHVYWLEMVLFYRAHFGTDETILDGKEYDIYVSYARNAEEEEFVLLTLRGVLENEFGYKLCIFDRDSLPGGIVTDETLSFIQKSRRLLVVLSPNYVLQGTQALLELKAGLENMASRGNINVILVQYKAVKETKVKELKRAKTVLTVIKWKGEKSKYPQGRFWKQLQVAMPVKKSPRRSSSDEQGLSYSSLKNV

SEQ ID NO: 19 - protein, 349 aa, synthetic construct
MLLLVTSLLLCELPHPAFLLIPEMWHEGLEEASRLYFGERNVKGMFEVLEPLHAMMERGPQTLKETSFNQAYGRDLMEAQEWCRKYMKSGNVKDLTQAWDLYYHVFRRISKSRKNPIDHHSIYCIIAVCSVFLMLINVLVIILKMFWIEATLLWRDIAKPYKTRNDGKLYDAYVVYPRNYKSSTDGASRVEHFVHQILPDVLENKCGYTLCIYGRDMLPGEDVVTAVETNIRKSRRHIFILTPQITHNKEFAYEQEVALHCALIQNDAKVILIEMEALSELDMLQAEALQDSLQHLMKVQGTIKWREDHIANKRSLNSKFWKHVRYQMPVPSKIPRKASSLTPLAAQKQ

SEQ ID NO: 20 - protein, 4 aa, synthetic construct
GGGS

SEQ ID NO: 21 - protein, 7 aa, synthetic construct
GGGSGGG

SEQ ID NO: 22 - protein, 5 aa, synthetic construct
  REGION 1..5: note = The entire sequence of amino acids 1-5 may be repeated one or more times.
GGGGS

SEQ ID NO: 23 - protein, 107 aa, synthetic construct
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 24 - protein, 89 aa, synthetic construct
EMWHEGLEEASRLYFGERNVKGMFEVLEPLHAMMERGPQTLKETSFNQAYGRDLMEAQEWCRKYMKSGNVKDLTQAWDLYYHVFRRISK

(19) United States
(12) Patent Application Publication
(10) Pub. No.: US 2025/0025502 A1
(43) Pub. Date: Jan. 23, 2025

(54) COMPOSITIONS AND METHODS FOR CONTROLLING CELL SIGNALING WITH CHIMERIC RECEPTORS

(71) Applicant: GentiBio, Inc., Cambridge, MA (US)

(72) Inventors: Tom Wickham, Scituate, MA (US); Philippe Kieffer-Kwon, Lexington, MA (US); Payam Zarin, Boston, MA (US)

(73) Assignee: GentiBio, Inc., Cambridge, MA (US)

(21) Appl. No.: 18/713,746

(22) PCT Filed: Nov. 29, 2022
(86) PCT No.: PCT/US2022/080582

§ 371 Date: May 28, 2024

Related U.S. Application Data
(60) Provisional application No. 63/283,973, filed on Nov. 29, 2021.
(60) Provisional application No. 63/283,977, filed on Nov. 29, 2021.

(51) Int. Cl. A61K 35/17; C07K 14/47; C07K 14/715; C12N 5/0783; C12N 9/12; C12N 9/90; C12N 15/86

(52) U.S. Cl. CPC A61K 35/17; C07K 14/4702; C07K 14/7155; C12N 5/0637; C12N 9/12; C12N 9/90; C12N 15/86; C07K 2319/03; C12N 2740/15043; C12N 2750/14143; C12Y 207/11001

(57) ABSTRACT

Described herein are compositions and methods for producing regulatory T cells (Tregs) with stable phenotypes by modifying cells to activate specific interleukin pathways.

### RELATED APPLICATIONS

This application claims the benefit under 35 U.S.C. § 119(e) of U.S. provisional application No. 63/283,973, filed Nov. 29, 2021, and U.S. provisional application No. 63/283,977, filed Nov. 29, 2021, each of which are incorporated by reference herein in their entirety.

### REFERENCE TO AN ELECTRONIC SEQUENCE LISTING

The contents of the electronic Sequence Listing (G097170022WO00-SEQ-JXV.xml; Size: 30,388 bytes; and Date of Creation: Nov. 29, 2022) are herein incorporated by reference in their entirety.

### BACKGROUND

Initiating or maintaining cell signaling to achieve a particular cellular function is useful for numerous therapeutic purposes, e.g., to initiate or maintain a particular cellular function or phenotype in therapeutic cells.

### SUMMARY

The disclosure, in some aspects, provides a chimeric signaling receptor for controlling ST2 signaling in a cell, the chimeric signaling receptor comprising a first polypeptide and a second polypeptide, wherein: the first polypeptide comprises a first extracellular domain, a first transmembrane domain, and a first membrane proximal signaling domain, the second polypeptide comprises a second extracellular domain, a second transmembrane domain, and a second membrane proximal signaling domain, the first extracellular domain comprises the rapamycin binding domain of FK-binding protein 12 (FKBP) or a functional fragment thereof, the second extracellular domain comprises the rapamycin binding domain of FKBP12-Rapamycin Binding domain of mTOR (FRB) or functional fragment thereof; and wherein (i) the first membrane proximal signaling domain comprises an intracellular signaling domain or functional fragment of ST2, and the second membrane proximal signaling domain comprises an intracellular signaling domain or functional fragment of IL1RAP; or (ii) the first membrane proximal signaling domain comprises an intracellular signaling domain or functional fragment of IL1RAP, and the second membrane proximal signaling domain comprises an intracellular signaling domain or functional fragment of ST2.

The disclosure, in some aspects, provides a chimeric signaling receptor for controlling IL18R signaling in a cell, the chimeric signaling receptor comprising a first polypeptide and a second polypeptide, wherein: the first polypeptide comprises a first extracellular domain, a first transmembrane domain, and a first membrane proximal signaling domain, the second polypeptide comprises a second extracellular domain, a second transmembrane domain, and a second membrane proximal signaling domain, the first extracellular domain comprises the rapamycin binding domain of FK-binding protein 12 (FKBP) or a functional fragment thereof, the second extracellular domain comprises the rapamycin binding domain of FKBP12-Rapamycin Binding domain of mTOR (FRB) or functional fragment thereof; and wherein (i) the first membrane proximal signaling domain comprises an intracellular signaling domain or functional fragment of IL18R1, and the second membrane proximal signaling domain comprises an intracellular signaling domain or functional fragment of IL18RAP; or (ii) the first membrane proximal signaling domain comprises an intracellular signaling domain or functional fragment of IL18RAP, and the second membrane proximal signaling domain comprises an intracellular signaling domain or functional fragment of IL18R1.

The disclosure, in some aspects, provides a chimeric signaling receptor for controlling IL36R signaling in a cell, the chimeric signaling receptor comprising a first polypeptide and a second polypeptide, wherein: the first polypeptide comprises a first extracellular domain, a first transmembrane domain, and a first membrane proximal signaling domain, the second polypeptide comprises a second extracellular domain, a second transmembrane domain, and a second membrane proximal signaling domain, the first extracellular domain comprises the rapamycin binding domain of FK-binding protein 12 (FKBP) or a functional fragment thereof, the second extracellular domain comprises the rapamycin binding domain of FKBP12-Rapamycin Binding domain of mTOR (FRB) or functional fragment thereof; and wherein (i) the first membrane proximal signaling domain comprises an intracellular signaling domain or functional fragment of IL36R, and the second membrane proximal signaling domain comprises an intracellular signaling domain or functional fragment of IL1RAP; or (ii) the first membrane proximal signaling domain comprises an intracellular signaling domain or functional fragment of IL1RAP, and the second membrane proximal signaling domain comprises an intracellular signaling domain or functional fragment of IL36R.

The disclosure, in some aspects, provides a chimeric signaling receptor for controlling IL10R signaling in a cell, the chimeric signaling receptor comprising a first polypeptide and a second polypeptide, wherein: the first polypeptide comprises a first extracellular domain, a first transmembrane domain, and a first membrane proximal signaling domain, the second polypeptide comprises a second extracellular domain, a second transmembrane domain, and a second membrane proximal signaling domain, the first extracellular domain comprises the rapamycin binding domain of FK-binding protein 12 (FKBP) or a functional fragment thereof, the second extracellular domain comprises the rapamycin binding domain of FKBP12-Rapamycin Binding domain of mTOR (FRB) or functional fragment thereof; and wherein (i) the first membrane proximal signaling domain comprises an intracellular signaling domain or functional fragment of IL10R1, and the second membrane proximal signaling domain comprises an intracellular signaling domain or functional fragment of IL10R2; or (ii) the first membrane proximal signaling domain comprises an intracellular signaling domain or functional fragment of IL10R2, and the second membrane proximal signaling domain comprises an intracellular signaling domain or functional fragment of IL10R1.

The disclosure, in some aspects, provides a chimeric signaling receptor for controlling LIFR signaling in a cell, the chimeric signaling receptor comprising a first polypeptide and a second polypeptide, wherein: the first polypeptide comprises a first extracellular domain, a first transmembrane domain, and a first membrane proximal signaling domain, the second polypeptide comprises a second extracellular domain, a second transmembrane domain, and a second membrane proximal signaling domain, the first extracellular domain comprises the rapamycin binding domain of FK-binding protein 12 (FKBP) or a functional fragment thereof, the second extracellular domain comprises the rapamycin binding domain of FKBP12-Rapamycin Binding domain of mTOR (FRB) or functional fragment thereof; and wherein (i) the first membrane proximal signaling domain comprises an intracellular signaling domain or functional fragment of gp130, and the second membrane proximal signaling domain comprises an intracellular signaling domain or functional fragment of LIFrβ; or (ii) the first membrane proximal signaling domain comprises an intracellular signaling domain or functional fragment of LIFrβ, and the second membrane proximal signaling domain comprises an intracellular signaling domain or functional fragment of gp130.

The disclosure, in some aspects, provides a chimeric signaling receptor comprising a first polypeptide and a second polypeptide, wherein: the first polypeptide comprises a first extracellular domain, a first transmembrane domain, a first membrane proximal signaling domain, and a first membrane distal signaling domain, and the second polypeptide comprises a second extracellular domain, a second transmembrane domain, a second membrane proximal signaling domain, and a second membrane distal signaling domain.

In some embodiments, the first membrane proximate signaling domain comprises an intracellular signaling domain or functional fragment of ST2, the second membrane proximate signaling domain comprises an intracellular signaling domain or functional fragment of IL1RAP, the first membrane distal signaling domain comprises an intracellular signaling domain or functional fragment of IL2Rγ, and the second membrane distal signaling domain comprises an intracellular signaling domain or functional fragment of IL2Rβ.

In some embodiments, the chimeric signaling receptor further comprises an extracellular linker. In some aspects, the extracellular linker comprises (G4S)xN peptide linker or an extracellular hinge of IL1RI, IL1RII, IL1RAP, IL2Rβ, IL2Rγ, IL10R1, IL10R2, IL18R1, IL18RAP, ST2, IL36R, LIFrβ or gp130.

The disclosure, in some aspects, provides a chimeric signaling receptor comprising a first polypeptide and a second polypeptide, wherein the first polypeptide and the second polypeptide each comprise a sequence having at least 80%, at least 85%, at least 90%, at least 95%, at least 96%, at least 97%, at least 98%, at least 99% identity to a sequence selected from SEQ ID NOs: 1-19.

In some embodiments, the first polypeptide and the second polypeptide are each identical to a sequence selected from SEQ ID NOs: 1-19.

In some embodiments, the disclosure provides a nucleic acid encoding the first polypeptide or the second polypeptide of a chimeric signaling receptor described herein. In some embodiments, the nucleic acid encodes the first polypeptide and the second polypeptide. In some embodiments, the nucleic acid, further comprises a promoter that is operably linked to a coding sequence encoding the first polypeptide and/or the second polypeptide. In some embodiments, the promoter is a constitutive promoter. In some embodiments, the constitutive promoter is an EF-1α, a PGK promoter, or an MND promoter. In some embodiments, the promoter is an MND promoter. In some embodiments, the promoter is an inducible promoter. In some embodiments, the inducible promoter is inducible by a drug or steroid.

In some embodiments, the disclosure provides a vector comprising any one of the nucleic acids described herein. In some embodiments, the vector is a viral vector. In some embodiments, the vector is an adenovirus-associated virus (AAV) vector. In some embodiments, the AAV vector is derived from an AAV of serotype AAV1, AAV2, AAV3, AAV4, AAV5, AAV6, AAV7, AAV8, AAV9, AAV10, or AAV11. In some embodiments, the viral vector is a lentiviral vector. In some embodiments, the vector is a plasmid. In some embodiments, the vector is a bacterial artificial chromosome. In some embodiments, the vector is a human artificial chromosome.

In some embodiments, the disclosure provides a lipid nanoparticle comprising any one of the nucleic acids described herein or any one of the vectors described herein.

In some embodiments, the disclosure provides a cell comprising any one of the chimeric signaling receptors described herein or any one of the nucleic acids described herein. In some embodiments, the cell is a stem cell or T cell. In some embodiments, the cell is a CD3+, CD4+, or CD8+ T cell. In some embodiments, the cell is a Treg cell. In some embodiments, the cell is a FoxP3+ Treg cell. In some embodiments, the cell is CTLA-4+, LAG-3+, CD25+, CD39+, CD27+, CD70+, GITR+, neuropilin-1+, galectin-1+, and/or IL-2Rα+. In some embodiments, the cell promotes Treg expansion. In some embodiments, the cell has an ST2 phenotype. In some embodiments, the cell has a Tr1 phenotype.

In some embodiments, the disclosure provides a pharmaceutical composition comprising any one of the cells described herein and rapamycin or a rapalog.

In some embodiments, the disclosure provides a method comprising administering to a subject any one of the pharmaceutical compositions described herein or any one of the cells described herein.

In some embodiments, the subject has or is at risk of developing an inflammatory disease, autoimmune disease, allergic disease, or a condition associated with a solid organ transplant. In some embodiments, the inflammatory disease is selected from pancreatic islet cell transplantation, asthma, hepatitis, traumatic brain injury, primary sclerosing cholangitis, primary biliary cholangitis, polymyositis, stroke, Still's disease, acute respiratory distress syndrome (ARDS), uveitis, inflammatory bowel disease (IBD), ulcerative colitis, graft-versus-host disease (GvHD), tolerance induction for transplantation, transplant rejection, or sepsis.

In some embodiments, the autoimmune disease is type 1 diabetes mellitus, multiple sclerosis, systemic lupus erythematosus, myasthenia gravis, rheumatoid arthritis, early onset rheumatoid arthritis, ankylosing spondylitis, immune-mediated pregnancy loss, immune-mediated recurrent pregnancy loss, dermatomyositis, psoriatic arthritis, Crohn's disease, inflammatory bowel disease (IBD), ulcerative colitis, bullous pemphigoid, pemphigus vulgaris, autoimmune hepatitis, psoriasis, Sjogren's syndrome, or celiac disease.

In some embodiments, the allergic disease is allergic asthma, steroid-resistant asthma, atopic dermatitis, celiac disease, pollen allergy, food allergy, drug hypersensitivity, or contact dermatitis.

In some embodiments, the condition associated with a solid organ transplant is graft-versus-host disease.

In some embodiments, the cell is autologous to the subject. In some embodiments, the cell is an allogeneic cell.

In some embodiments, the disclosure provides a method of producing an engineered cell, the method comprising introducing into the cell any one of the nucleic acids described herein.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1A illustrates a chimeric signaling receptor comprising a first polypeptide and a second polypeptide, wherein the first polypeptide comprises a first extracellular domain, a first transmembrane domain, and a first membrane proximal signaling domain, and the second polypeptide comprises a second extracellular domain, a second transmembrane domain, and a second membrane proximal signaling domain.

FIG. 1B illustrates a chimeric signaling receptor comprising a first polypeptide and a second polypeptide, wherein the first polypeptide comprises a first extracellular domain, a first transmembrane domain, a first membrane proximal signaling domain, and a first membrane distal domain, and the second polypeptide comprises a second extracellular domain, a second transmembrane domain, a second membrane proximal signaling domain, and a second membrane distal signaling domain. A chimeric signaling receptor having two signaling domains on each of the polypeptides allows for initiation and/or maintenance of two cell signaling pathways within the cells.

FIG. 2 illustrates examples of chimeric signaling receptors with different configurations of signaling domains. CISC=chemical-inducible signaling complex.

FIG. 3 provides examples of polypeptides that form the chimeric signaling ST2.2 receptor depicted in FIG. 2. Each polypeptide comprises an extracellular domain comprising FRB or FKBP, a transmembrane domain (“TMD”), a membrane proximal signaling domain, a membrane distal linker and a membrane distal signaling domain.

FIG. 4A illustrates examples of chimeric signaling receptors with different configurations of signaling domains and transmembrane domains.

FIG. 4B provides the results of a secreted embryonic alkaline phosphatase (SEAP) colorimetric assay, demonstrating that murinized CISC2 signals through the IL2:STAT5 signaling axis. Cells comprising the different CISC receptors shown in FIG. 4A were stimulated with IL2 in the presence and absence of rapamycin.

FIG. 5 provides examples of polypeptides that may form a chimeric signaling RISE33 (RISE ST2) receptor as illustrated in FIG. 2. Each polypeptide comprises an extracellular domain comprising FRB or FKBP, a transmembrane domain (“TMD”), a membrane proximal signaling domain, a membrane distal linker, and a membrane distal signaling domain.

FIG. 6A provides the results of a SEAP colorimetric assay demonstrating humanized CISC 33 (RISE ST2) signaling through the IL33 signaling pathway. Cells were treated with either IL33 or rapamycin at the doses shown.

FIG. 6B provides the results of a SEAP colorimetric assay demonstrating murinized CISC 33 (RISE ST2) signaling through the IL33 signaling pathway. Cells were treated with either recombinant IL33 or rapamycin at varying doses.

FIG. 7 provides examples of polypeptides that may form the chimeric signaling RISE10 receptor as illustrated in FIG. 2. Each polypeptide comprises an extracellular domain comprising FRB or FKBP, a transmembrane domain (“TMD”), a membrane proximal signaling domain, a membrane distal linker and a membrane distal signaling domain.

FIG. 8 provides an example of CISC signaling through the IL10 signaling pathway. Cells were treated with IL10 or rapamycin at varying doses.

FIGS. 9A-9H provide examples of amino acid sequences of CISC polypeptides.

FIG. 10 shows exemplary flow cytometry results of T cells (HEK293, human CD4 and hEngTregs) transfected with RISE33 constructs.

FIG. 11 shows exemplary flow cytometry results of CD4 T cells and Jurkat cells transfected with RISE33 (or mock-transfected) and stimulated under different conditions. CD69 is a marker of T cell activation.

### DETAILED DESCRIPTION

Initiating or maintaining cell signaling to achieve a particular cellular function or phenotype in target cells in vivo without affecting other cells is difficult, at least because it is difficult to control the destination of ligands that might be used to initiate or maintain the cell signaling. For example, administering of ligands to control cell signaling in particular target cells can have significant on-target, but off-tissue or off-cell effects that may be undesirable. This disclosure provides a solution for overcoming this difficulty in cases where the target cells (e.g., T cells such as CAR-T cells) are administered to a subject in which cell signaling is to be controlled.

Provided herein are compositions and methods for utilizing a chimeric signaling receptor to be expressed in target cells that are to be administered to a subject, such that administering of a ligand to the subject initiates or maintains a particular cell signaling in the target cells. The particular cell signaling is controlled by contacting cells expressing the chimeric signaling receptor with a ligand that binds to extracellular domains and activates the chimeric receptor. In some embodiments, the binding of a ligand promotes dimerization of a first polypeptide of a chimeric receptor and a second polypeptide of a chimeric receptor via dimerization of a first extracellular domain in the first polypeptide and a second extracellular domain in the second polypeptide. The complex, comprising the first polypeptide and the second polypeptide, is referred to herein as a chemical-inducible signaling complex (CISC) or, in some embodiments, a rapamycin induced signal enhancement (RISE) complex. The particular cell signaling that is controlled is governed by intracellular signaling domains of the chimeric receptor that once activated by a ligand binding to extracellular domains of the receptor, triggers the particular cell signaling. The particular cell signaling may be signaling involved in any cellular function, such as proliferation, cellular division and replication, DNA replication, protein synthesis, cytokine production, maintenance of a cytotoxic phenotype (e.g., as observed in cytotoxic T cells), cell homing, maintenance of a regulatory phenotype (e.g., as observed in regulatory T cells), antigen recognition and activation, or tissue repair.

### Chimeric Signaling Receptors

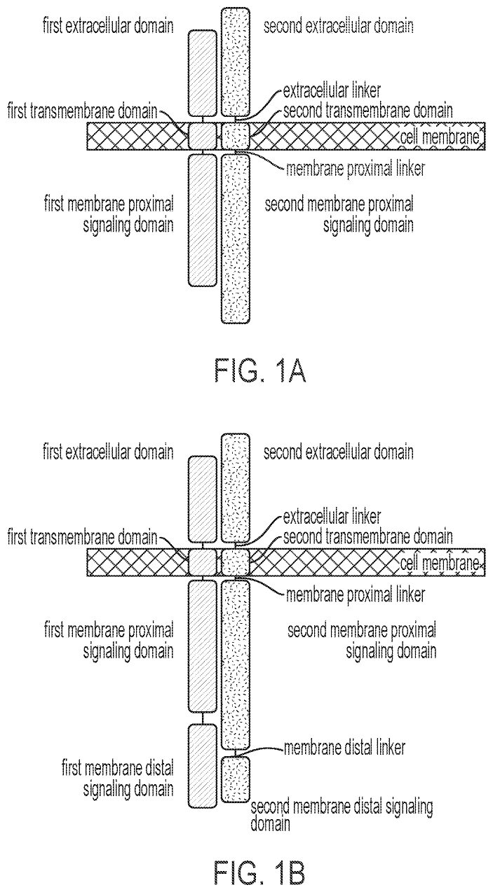

In some aspects, provided herein is a chimeric signaling receptor comprising a first polypeptide and a second polypeptide, wherein the first polypeptide comprises a first extracellular domain, a first transmembrane domain, and a first membrane proximal signaling domain, and the second polypeptide comprises a second extracellular domain, a second transmembrane domain, and a second membrane proximal signaling domain, such that the first and second extracellular binding domains are capable of binding a ligand (e.g., a small molecule such as rapamycin or an analog thereof). A non-limiting schematic of this receptor is provided in FIG. 1A.

In some embodiments, the first polypeptide of chimeric signaling receptor further comprises a first membrane distal signaling domain and the second polypeptide of a chimeric signaling receptor further comprises a second distal signaling domain. A non-limiting schematic of this receptor is provided in FIG. 1B.

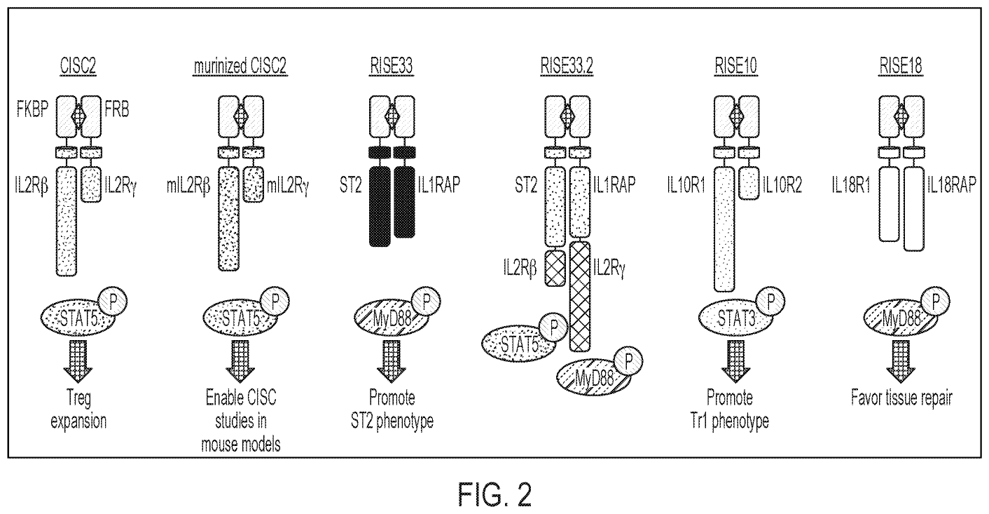

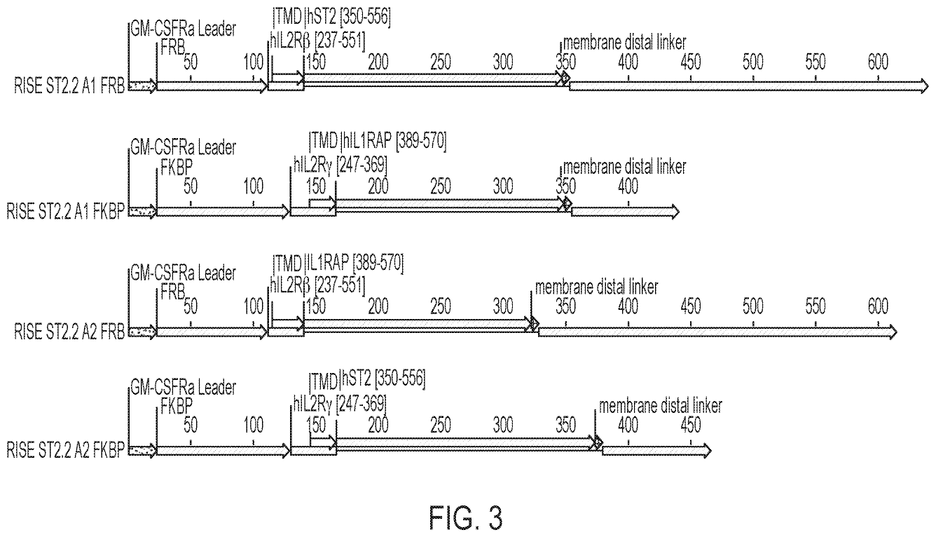

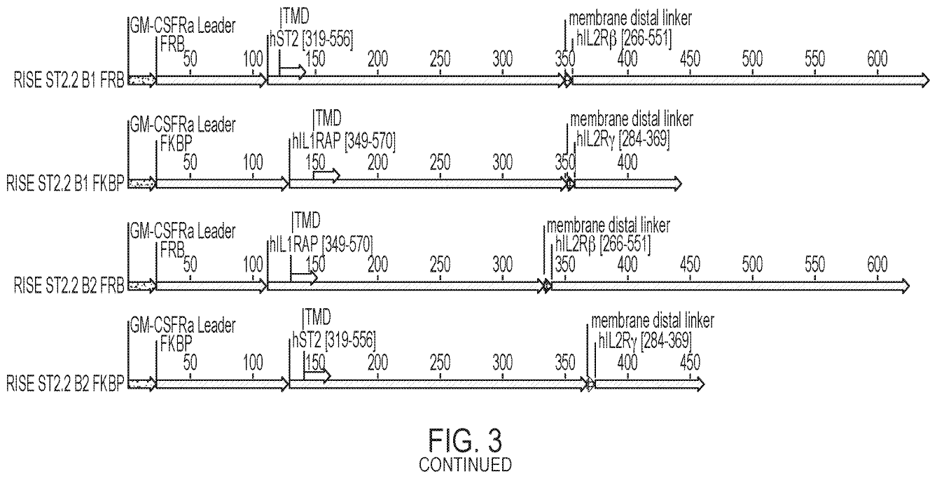

FIG. 2 provides examples of a chimeric signaling receptor as provided herein that are activatable by rapamycin or a rapalog and will be described in more detail below.

In some embodiments, a polypeptide of a chimeric signaling receptor (e.g., first polypeptide or a second polypeptide) further comprises an extracellular linker between an extracellular domain and a transmembrane domain. In some embodiments, a polypeptide of a chimeric signaling receptor (e.g., first polypeptide or a second polypeptide) further comprises a membrane proximal linker between a transmembrane domain and a membrane proximal signaling domain. In some embodiments, a polypeptide of a chimeric signaling receptor (e.g., first polypeptide or a second polypeptide) further comprises a membrane distal linker between a membrane proximal signaling domain and a membrane distal signaling domain. In some embodiments, a chimeric signaling receptor as provided herein comprises more than one linker. In some embodiments, a polypeptide of a chimeric signaling receptor further comprises a signal peptide.

In some embodiments, the first extracellular domain and second extracellular domains are dimerization domains that dimerize in the presence of a ligand. In some embodiments, a first extracellular domain is the same as a second extracellular domain and the first and second extracellular domains homodimerize upon binding to a ligand. In some embodiments, a first extracellular domain is different from a second extracellular domain and the first and second extracellular domains heterodimerize upon binding to a ligand. The ligand may be a small molecule, peptide, protein, or other biologic (e.g., comprising base pairs). In some embodiments, a ligand that activates a chimeric signaling receptor as provided herein is rapamycin or a rapalog. Rapamycin, a macrocyclic triene compound, is also known as sirolimus and is commercially available (e.g., RAPAMUNE®). In some embodiments, the ligand that activates a chimeric signaling receptor is wild-type rapamycin (e.g., commercially available rapamycin, such as RAPAMUNE®). In some embodiments, the ligand that activates a chimeric signaling receptor comprises rapamycin having 1, 2, 3, 4, 5, or more modifications relative to wild-type rapamycin. For example, in some embodiments, the methyl side group chain of the rapamycin may be modified (e.g., extended). In some embodiments, the rapalog is everolimus, CCI-779, C20-methallylrapamycin, C16-(S)-3-methylindolerapamycin, C16-iRap, C16-(S)-7-methylindolerapamycin, AP21967, C16-(S)Butylsulfonamidorapamycin, AP23050, sodium mycophenolic acid, benidipine hydrochloride, AP1903, AP23573, or a metabolite or derivative thereof.

In some embodiments, the first extracellular domain comprises the rapamycin binding domain of FK-binding protein 12 (FKBP), and the second extracellular domain comprises the rapamycin binding domain of FKBP12-Rapamycin Binding domain of mTOR (FRB). In some embodiments, the sequence of the FKBP domain is: GVQVETISPGDGRTFPKRGQTCVVHYTGMLEDGKKFDSSRDRNKPFKFMLGKQEVI RGWEEGVAQMSVGQRAKLTISPDYAYGATGHPGIIPPHATLVFDVELLKLE (SEQ ID NO: 23). In some embodiments, the FKBP domain may comprise a polypeptide having at least 50% identity (e.g., at least 50, at least 60, at least 70, at least 80, at least 90, at least 95, at least 98, at least 99, or at least 99.5% identity) to SEQ ID NO: 23. In some embodiments, the FKBP domain comprises up to 20 amino acid substitutions, up to 15 amino acid sequence substitutions, up to 10 amino acid substitutions. In some embodiments, the FKBP domain comprises 1, 2, 3, 4, 5, 6, 7, 8, 9, 10, or more amino acid sequence substitutions.

In some embodiments, the sequence of the FRB domain is EMWHEGLEEASRLYFGERNVKGMFEVLEPLHAMMERGPQTLKETSFNQAYGRDLM EAQEWCRKYMKSGNVKDLTQAWDLYYHVFRRISK (SEQ ID NO; 24). In some embodiments, the FRB domain may comprise a polypeptide having at least 50% identity (e.g., at least 50, at least 60, at least 70, at least 80, at least 90, at least 95, at least 98, at least 99, or at least 99.5% identity) to SEQ ID NO: 24. In some embodiments, the FRB domain comprises up to 20 amino acid substitutions, up to 15 amino acid sequence substitutions, up to 10 amino acid substitutions. In some embodiments, the FRB domain comprises 1, 2, 3, 4, 5, 6, 7, 8, 9, 10, or more amino acid sequence substitutions.

In one embodiment, the FRB domain comprises an T74L mutation. In some embodiments, a first extracellular domain of a chimeric signaling receptor comprises a functional fragment of FKBP, the rapamycin binding domain of FK-binding protein 12. In some embodiments, a second extracellular domain of a chimeric signaling receptor comprises a functional fragment of FRB, the rapamycin binding domain of FKBP12-Rapamycin Binding domain of mTOR. A functional fragment of FKBP or FRB is a fragment that dimerizes with a counterpart fragment upon binding to a ligand (e.g., rapamycin or a rapalog).

In some embodiments, a chimeric signaling receptor as provided herein controls signaling that is instigated, propagated, or maintained by an interleukin (IL). In some embodiments, the IL is a member of the IL1 superfamily. An IL in the IL1 superfamily may include ILs in any subfamily, e.g., ILs that share IL-1RAP as their secondary receptor (e.g., IL1, IL33, or IL36), or IL18. In some embodiments, a signaling domain of a chimeric signaling receptor (e.g., a first membrane proximal signaling domain, second membrane proximal signaling domain, a first membrane distal signaling domain, or second membrane distal signaling domain) comprises an intracellular signaling domain or functional fragment of a receptor for an IL1 superfamily member (e.g., IL1, IL18, IL33, IL36, IL37, or IL38). Fields et al. (Front Immunol, 2019 Jun. 20; 10:1412) provides a description of IL1 superfamily signaling and receptors thereof, and is incorporated herein by reference in its entirety.

In some embodiments, a first membrane proximal signaling domain comprises an intracellular signaling domain or functional fragment of IL1R (e.g., IL1RI, IL1RII, or IL1RAP), IL2Rβ, IL2Rγ, IL10R1, IL10R2, IL18R1, IL18RAP, ST2, IL36R, LIFR, or gp130.

In some embodiments, a second membrane proximal signaling domain comprises an intracellular signaling domain or functional fragment of IL1R (e.g., IL1RI, IL1RII, or IL1RAP), IL2Rβ, IL2Rγ, IL10R1, IL10R2, IL18R1, IL18RAP, ST2, IL36R, LIFR, or gp130.

In some embodiments, a first membrane proximal signaling domain comprises an intracellular signaling domain or functional fragment of IL2Rβ (e.g., of SEQ ID NOs: 5, 7), and a second membrane proximal signaling domain comprises an intracellular signaling domain or functional fragment of IL2Rγ (e.g., of SEQ ID NOs: 8, 10). In some embodiments, a first membrane proximal signaling domain comprises an intracellular signaling domain or functional fragment of IL2Rγ (e.g., of SEQ ID NOs: 4, 6), and a second membrane proximal signaling domain comprises an intracellular signaling domain or functional fragment of IL2Rβ (e.g., of SEQ ID NOs: 9, 11). In some embodiments, a first transmembrane domain comprises a transmembrane domain of IL2Rβ, and a second transmembrane domain comprises a transmembrane domain of IL2Rγ. In some embodiments, a first transmembrane domain and a second transmembrane domain each comprise a transmembrane domain of IL2Rβ. In some embodiments, a first transmembrane domain and a second transmembrane domain each comprise a transmembrane domain of IL2Rγ.

In some embodiments, a first membrane proximal signaling domain comprises an intracellular signaling domain or functional fragment of ST2 (e.g., of SEQ ID NOs: 9, 10), and a second membrane proximal signaling domain comprises an intracellular signaling domain or functional fragment of IL1RAP (e.g., of SEQ ID NOs: 4, 7). In some embodiments, a first membrane proximal signaling domain comprises an intracellular signaling domain or functional fragment of IL1RAP (e.g., of SEQ ID NOs: 8, 11), and a second membrane proximal signaling domain comprises an intracellular signaling domain or functional fragment of ST2 (e.g., of SEQ ID NOs: 5, 6). In some embodiments, a first membrane distal signaling domain comprises an intracellular signaling domain or functional fragment of IL2Rβ (e.g., of SEQ ID NOs: 9, 11), and a second membrane distal signaling domain comprises an intracellular signaling domain or functional fragment of IL2Rγ (e.g., of SEQ ID NOs: 8, 10). In some embodiments, a first membrane distal signaling domain comprises an intracellular signaling domain or functional fragment of IL2Rγ (e.g., of SEQ ID NOs: 8, 10), and a second membrane distal signaling domain comprises an intracellular signaling domain or functional fragment of IL2Rβ (e.g., of SEQ ID NOs: 9, 11).

In some embodiments, a first membrane proximal signaling domain comprises an intracellular signaling domain or functional fragment of IL36R, and a second membrane proximal signaling domain comprises an intracellular signaling domain or functional fragment of IL1RAP (e.g., of SEQ ID NO: 4). In some embodiments, a first membrane proximal signaling domain comprises an intracellular signaling domain or functional fragment of IL1RAP (e.g., of SEQ ID NO: 4), and a second membrane proximal signaling domain comprises an intracellular signaling domain or functional fragment of IL36R.

In some embodiments, a first membrane proximal signaling domain comprises an intracellular signaling domain or functional fragment of IL18R1, and a second membrane proximal signaling domain comprises an intracellular signaling domain or functional fragment of IL18RAP. In some embodiments, a first membrane proximal signaling domain comprises an intracellular signaling domain or functional fragment of IL18RAP, and a second membrane proximal signaling domain comprises an intracellular signaling domain or functional fragment of IL18R1.

In some embodiments, a first membrane proximal signaling domain comprises an intracellular signaling domain or functional fragment of IL10R1 (e.g., of SEQ ID NOs: 13, 15), and a second membrane proximal signaling domain comprises an intracellular signaling domain or functional fragment of IL10R2 (e.g., of SEQ ID NOs; 12, 14). In some embodiments, a first membrane proximal signaling domain comprises an intracellular signaling domain or functional fragment of IL10R2, and a second membrane proximal signaling domain comprises an intracellular signaling domain or functional fragment of IL10R1.

In some embodiments, a first membrane proximal signaling domain comprises an intracellular signaling domain or functional fragment of gp130, and a second membrane proximal signaling domain comprises an intracellular signaling domain or functional fragment of LIFrβ. In some embodiments, a first membrane proximal signaling domain comprises an intracellular signaling domain or functional fragment of LIFrβ, and a second membrane proximal signaling domain comprises an intracellular signaling domain or functional fragment of gp130.

In some embodiments, a first membrane distal signaling domain comprises an intracellular signaling domain or functional fragment of IL1R (e.g., IL1RI, IL1RII, or IL1RAP), IL2Rβ, IL2Rγ, IL10R1, IL10R2, IL18R1, IL18RAP, ST2, IL36R, LIFR or gp130. In some embodiments, a second membrane distal signaling domain comprises an intracellular signaling domain or functional fragment of IL1R (e.g., IL1RI, IL1RII, or IL1RAP), IL2Rβ, IL2Rγ, IL10R1, IL10R2, IL18R1, IL18RAP, ST2, IL36R, LIFR or gp130.

In some embodiments, a first membrane proximal signaling domain and second membrane proximal distancing domain are linked (e.g., using extracellular and/or membrane proximal linkers of comparable size) respectively to the first extracellular domain and second extracellular domain such that the signaling domains can effectuate downstream signaling (e.g., phosphorylation of Jak1 and Jak3 in the case of IL2R signaling). In some embodiments, a first membrane distal signaling domain and second membrane distal distancing domain are further linked (e.g., using membrane distal linkers of comparable size) respectively to the first extracellular domain (and first membrane proximal domain) and second extracellular domain (and second membrane proximal domain) such that all four signaling domains can effectuate downstream signaling (e.g., phosphorylation of Jak1 and Jak3 in the case of IL2R signaling, and activation of MyD88).

The chimeric signaling receptor of the present disclosure may have any configuration of first and second membrane proximal signaling domains with first and second membrane proximal signaling domains. For example, the first and second membrane proximal signaling domains may provide IL2R signaling while the first and second membrane distal signaling domains may provide ST2 signaling. As another non-limiting example, the first and second membrane proximal signaling domains may provide ST2 signaling while the first and second membrane distal signaling domains may provide IL2 signaling. Further, the membrane proximal and distal binding domains may be linked with any extracellular domain. For example, a first polypeptide comprising IL2Rβ and ST2 signaling domains may further comprise FKBP, and a second polypeptide comprising IL2Rγ and IL1RAP signaling domains may further comprise FRB. Alternatively, a first polypeptide comprising IL2Rβ and ST2 signaling domains may further comprise FRB, and a second polypeptide comprising IL2Rγ and IL1RAP signaling domains may further comprise FKBP.

In some embodiments, a first membrane distal signaling domain comprises an intracellular signaling domain or functional fragment of IL2Rβ, and a second membrane distal signaling domain comprises an intracellular signaling domain or functional fragment of IL2Rγ. In some embodiments, a first membrane distal signaling domain comprises an intracellular signaling domain or functional fragment of IL2Rγ, and a second membrane distal signaling domain comprises an intracellular signaling domain or functional fragment of IL2R3.

In some embodiments, a first membrane distal signaling domain comprises an intracellular signaling domain or functional fragment of ST2, and a second membrane distal signaling domain comprises an intracellular signaling domain or functional fragment of IL1RAP. In some embodiments, a first membrane distal signaling domain comprises an intracellular signaling domain or functional fragment of IL1RAP, and a second membrane distal signaling domain comprises an intracellular signaling domain or functional fragment of ST2.

In some embodiments, a first membrane distal signaling domain comprises an intracellular signaling domain or functional fragment of IL36R, and a second membrane distal signaling domain comprises an intracellular signaling domain or functional fragment of IL1RAP. In some embodiments, a first membrane distal signaling domain comprises an intracellular signaling domain or functional fragment of IL1RAP, and a second membrane distal signaling domain comprises an intracellular signaling domain or functional fragment of IL36R.

In some embodiments, a first membrane distal signaling domain comprises an intracellular signaling domain or functional fragment of IL18R1, and a second membrane distal signaling domain comprises an intracellular signaling domain or functional fragment of IL18RAP. In some embodiments, a first membrane distal signaling domain comprises an intracellular signaling domain or functional fragment of IL18RAP, and a second membrane distal signaling domain comprises an intracellular signaling domain or functional fragment of IL18R1.

In some embodiments, a first membrane distal signaling domain comprises an intracellular signaling domain or functional fragment of IL10R1, and a second membrane distal signaling domain comprises an intracellular signaling domain or functional fragment of IL10R2. In some embodiments, a first membrane distal signaling domain comprises an intracellular signaling domain or functional fragment of IL10R2, and a second membrane distal signaling domain comprises an intracellular signaling domain or functional fragment of IL10R1.

In some embodiments, a first membrane distal signaling domain comprises an intracellular signaling domain or functional fragment of gp130, and a second membrane distal signaling domain comprises an intracellular signaling domain or functional fragment of LIFrβ. In some embodiments, a first membrane distal signaling domain comprises an intracellular signaling domain or functional fragment of LIFrβ, and a second membrane distal signaling domain comprises an intracellular signaling domain or functional fragment of gp130.

In some embodiments, a transmembrane domain (e.g., a first transmembrane domain or a second transmembrane domain) comprises a transmembrane domain or fragment of a transmembrane domain of IL1R (e.g., IL1RI, IL1RII, or IL1RAP), IL2Rβ, IL2Rγ, IL10R1, IL10R2, IL18R1, IL18RAP, ST2, IL36R, LIFR or gp130. In some embodiments, a transmembrane domain of IL2Rβ, IL2Rγ, IL10R1, IL10R2, IL18R1, IL18RAP, ST2, IL1RAP, LIFR or gp130 comprises a part that extends in the cytosol of a native cell is which it is expressed, and/or a part that extends in the extracellular space relative to a native cell in which it is expressed. In some embodiments, a polypeptide of a chimeric signaling receptor (e.g., a first or second polypeptide) comprises a transmembrane domain or fragment thereof and an intracellular signaling domain or functional fragment thereof of the same receptor polypeptide (e.g., a transmembrane domain and intracellular signaling domain of ST2). In some embodiments, a polypeptide of a chimeric signaling receptor (e.g., a first or second polypeptide) comprises a transmembrane domain or fragment thereof and an intracellular signaling domain or functional fragment thereof of a different receptor polypeptide (e.g., a transmembrane domain of IL1RAP and intracellular signaling domain of ST2).

In some embodiments, an extracellular linker that links an extracellular domain and a transmembrane domain (e.g., a first or second extracellular linker) comprises a peptide (e.g., a human or artificial peptide). In some embodiments, an extracellular linker that links an extracellular domain and a transmembrane domain (e.g., the first or second extracellular linker) comprises a synthetic linker (e.g., a flexible linker) or an extracellular hinge domain. In some embodiments, the linker is a flexible linker or a rigid linker. In some embodiments, the linker is a flexible linker. In some embodiments, the linker comprises 1, 2, 3, 4, 5, 6, 7, 8, 9 or 10 amino acids, such as Gly, or a number of amino acids, such as Gly, within a range defined by any two of the aforementioned numbers. In some embodiments, the Gly spacer comprises at least 3 Gly. In some embodiments, the Gly spacer comprises a sequence set forth as GGGS (SEQ ID NO: 20), GGGSGGG (SEQ ID NO: 21) or GGG. In principle, to provide flexibility, the flexible linkers generally comprise small, non-polar (e.g., Gly) or polar (e.g., Ser or Thr) amino acids, such an underlying sequence of alternating Gly and Ser residues. Solubility of the linker and associated chimeric signaling receptor may be enhanced by including charged residues; e.g., two positively charged residues (Lys) and one negatively charged residue (Glu). The linker may vary from 2 to 31 amino acids, optimized for each condition so that the linker does not impose any constraints on the conformation or interactions of the linked partners in lengths, such as between 12 and 18 residues.

In some embodiments, an extracellular binding domain may be connected to a transmembrane domain via a hinge domain (“hinge”). A hinge refers to a domain that links the extracellular binding domain to the transmembrane domain and may confer flexibility to the extracellular binding domain. In some embodiments, the hinge domain positions the extracellular domain close to the plasma membrane to minimize the potential for recognition by antibodies or binding fragments thereof. In some embodiments, the hinge domain may be natural or synthetic.

In some embodiments, an extracellular linker that links an extracellular domain and a transmembrane domain (e.g., a first or second extracellular linker) comprises (G4S)xN (SEQ ID NO: 22) peptide linker (where N may be 1, 2, 3, 4, 5, 6, 7, 8, 9, or 10 or more) or an extracellular hinge of IL1R (e.g., IL1RI, IL1RII, or IL1RAP), IL2Rβ, IL2Rγ, IL10R1, IL10R2, IL18R1, IL18RAP, ST2, IL38R, LIFrβ, or gp130. In some embodiments, the first extracellular hinge domain is from IL10R1 and the second extracellular hinge domain is from IL10R2.

In some embodiments, a membrane proximal linker that links a transmembrane domain and membrane proximate signaling domain (e.g., a first or second extracellular linker) comprises a peptide (e.g., a human or artificial peptide). In some embodiments, a membrane proximal linker that links a transmembrane domain and membrane proximate signaling domain (e.g., a first or second extracellular linker) comprises (G4S)xN (SEQ ID NO: 22) peptide linker (where N may be 1, 2, 3, 4, 5, 6, 7, 8, 9, or 10 or more) or an intracellular hinge/domain of IL1R (e.g., IL1RI, IL1RII, or IL1RAP), IL2Rβ, IL2Rγ, IL10R1, IL10R2, IL18R1, IL18RAP, ST2, IL36R, LIFrβ, or gp130.

In some embodiments, a membrane distal linker that links a membrane proximate signaling domain and membrane distal signaling domain (e.g., a first or second extracellular linker) comprises a peptide (e.g., a human or artificial peptide). In some embodiments, a membrane distal linker that links a membrane proximate signaling domain and membrane distal signaling domain (e.g., a first or second extracellular linker) comprises (G4S)xN (SEQ ID NO: 22) peptide linker (where N may be 1, 2, 3, 4, 5, 6, 7, 8, 9, or 10 or more) or an intracellular hinge/domain of IL1R (e.g., IL1RI, IL1RII, or IL1RAP), IL2Rβ, IL2Rγ, IL10R1, IL10R2, IL18R1, IL18RAP, ST2, IL36R, LIFrβ or gp130.

In some embodiments, any of the extracellular domain, transmembrane domain, membrane proximal signaling domain, membrane distal signaling domain, extracellular linker, membrane proximal linker, and membrane distal linker of a polypeptide of a chimeric signaling receptor is derived from a human protein. In some embodiments, any of the extracellular domain, transmembrane domain, membrane proximal signaling domain, membrane distal signaling domain, extracellular linker, membrane proximal linker, and membrane distal linker of a polypeptide of a chimeric signaling receptor is derived from a non-human source, e.g., murine proteins. In some embodiments, any of the extracellular domain, transmembrane domain, membrane proximal signaling domain, membrane distal signaling domain, extracellular linker, membrane proximal linker, and membrane distal linker of a polypeptide of a chimeric signaling receptor is derived from the same native receptor polypeptide.

In some aspects, provided herein is a chimeric signaling receptor for controlling cell signaling involved in proliferation, cellular division and replication, DNA replication, protein synthesis, cytokine production, maintenance of a cytotoxic phenotype (e.g., as observed in cytotoxic T cells), maintenance of a regulatory phenotype (e.g., as observed in regulatory T cells), cell homing, antigen recognition and activation, or tissue repair, e.g., in repair regulatory T (Treg) cells. In some embodiments, a chimeric signaling receptor comprises signaling domains that are involved in signaling after receptor binding of IL1 to IL1R or activation of IL1R receptor signaling after receptor binding of IL33 to ST2 or activation of ST2 receptor, signaling after receptor binding of IL18 to IL18 receptor or IL18 receptor activation, signaling after receptor binding of IL36 to IL36R or activation of IL36R receptor, signaling after receptor binding of IL2 to IL2 receptor or IL2 receptor activation, signaling after receptor binding of IL10 to IL10 receptor or IL10 receptor activation, signaling after receptor binding of LIF to LIF receptor or LIF receptor activation.

In some embodiments, the IL2 receptor is activated, resulting in IL2 signaling, leading to phosphorylation of STAT5 and expansion of Tregs. Moreover, IL2 signaling plays a role in immune homeostasis, and dysregulation of IL2 signaling has been identified as a factor in autoimmune diseases and immunodeficiency. In some embodiments a chimeric signaling receptor for controlling IL2 signaling comprises a first polypeptide and a second polypeptide, wherein the first polypeptide comprises a first extracellular domain, a first transmembrane domain, and a first membrane proximal signaling domain, and the second polypeptide comprises a second extracellular domain, a second transmembrane domain, and a second membrane proximal signaling domain.

In some embodiments, a first extracellular domain comprises FKBP or a functional fragment thereof and a second extracellular domain comprises FRB or a functional fragment thereof. In some embodiments, a first membrane proximal signaling domain comprises an intracellular signaling domain or functional fragment of IL2Rβ, and a second membrane proximal signaling domain comprises an intracellular signaling domain or functional fragment of IL2Rγ. In some embodiments, a first membrane proximal signaling domain comprises an intracellular signaling domain or functional fragment of IL2Rγ, and a second membrane proximal signaling domain comprises an intracellular signaling domain or functional fragment of IL2Rβ.

In some embodiments, a chimeric signaling receptor for controlling IL33 or ST2 signaling is provided herein. When bound to ST2, IL33 activates the MyD88, IRAK1, and TRAF6 pathways, resulting in downstream activation of the NF-κB, JNK, p38, and ERK signaling pathways. This activation enhances Foxp3 and GATA expression, Treg function, the expansion of ST2+ Tregs, and the production of type 2 cytokines (e.g., IL5 and IL13). IL33 or ST2+ Tregs are involved in repair of tissue (e.g., lung tissue), reduction of inflammation, and can migrate and proliferate in response to activation (e.g., by IL33). Accordingly, in some aspects, provided herein is a chimeric signaling receptor for controlling IL33 or ST2 signaling.

In some embodiments a chimeric signaling receptor for controlling ST2 signaling comprises a first polypeptide and a second polypeptide, wherein the first polypeptide comprises a first extracellular domain, a first transmembrane domain, and a first membrane proximal signaling domain, and the second polypeptide comprises a second extracellular domain, a second transmembrane domain, and a second membrane proximal signaling domain.

In some embodiments, a first extracellular domain comprises FKBP or a functional fragment thereof and a second extracellular domain comprises FRB or a functional fragment thereof.

In some embodiments, a first membrane proximate domain of a chimeric signaling receptor for controlling ST2 signaling comprises an intracellular signaling domain or functional fragment of ST2, and a second membrane proximate domain of a chimeric signaling receptor for controlling ST2 signaling comprises an intracellular signaling domain or functional fragment of IL1RAP. In some embodiments, a first membrane proximate domain of a chimeric signaling receptor for controlling ST2 signaling comprises an intracellular signaling domain or functional fragment of IL1RAP, and a second membrane proximate domain of a chimeric signaling receptor for controlling ST2 signaling comprises an intracellular signaling domain or functional fragment of ST2.

It is to be understood that any configuration of extracellular domains and signaling domains, including membrane proximate and membrane distal domains, on a first and second polypeptides of a chimeric signaling receptor are contemplated herein. In some embodiments, a chimeric signaling receptor comprises two signaling domains on each polypeptide. For example, a first and second membrane proximate signaling domains may include intracellular receptor domains of ST2, and a first and second membrane distal signaling domains may include intracellular receptor domains of IL2R. Alternatively, a chimeric signaling receptor may comprise a first and second membrane proximate signaling domains comprising intracellular receptor domains of IL2R, and a first and second membrane distal signaling domains comprising intracellular receptor domains of ST2.

In some embodiments, a chimeric signaling receptor for controlling ST2 signaling comprises a first membrane proximate signaling domain comprising an intracellular signaling domain or functional fragment of ST2, a second membrane proximate signaling domain comprising an intracellular signaling domain or functional fragment of IL1RAP, a first membrane distal signaling domain comprising an intracellular signaling domain or functional fragment of IL2Rβ, and a second membrane distal signaling domain comprising an intracellular signaling domain or functional fragment of IL2Rγ.

In some embodiments, a chimeric signaling receptor for controlling ST2 signaling comprises a first membrane proximate signaling domain comprising an intracellular signaling domain or functional fragment of IL1RAP, a second membrane proximate signaling domain comprising an intracellular signaling domain or functional fragment of ST2, a first membrane distal signaling domain comprising an intracellular signaling domain or functional fragment of IL2Rβ, and a second membrane distal signaling domain comprising an intracellular signaling domain or functional fragment of IL2Rγ.

In some embodiments, a chimeric signaling receptor for controlling ST2 signaling comprises a first membrane proximate signaling domain comprising an intracellular signaling domain or functional fragment of ST2, a second membrane proximate signaling domain comprising an intracellular signaling domain or functional fragment of IL1RAP, a first membrane distal signaling domain comprising an intracellular signaling domain or functional fragment of IL2Rγ, and a second membrane distal signaling domain comprising an intracellular signaling domain or functional fragment of IL2Rβ.

In some embodiments, a chimeric signaling receptor for controlling ST2 signaling comprises a first membrane proximate signaling domain comprising an intracellular signaling domain or functional fragment of IL1RAP, a second membrane proximate signaling domain comprising an intracellular signaling domain or functional fragment of ST2, a first membrane distal signaling domain comprising an intracellular signaling domain or functional fragment of IL2Rγ, and a second membrane distal signaling domain comprising an intracellular signaling domain or functional fragment of IL2Rβ.

In some embodiments, a chimeric signaling receptor for controlling IL18 signaling is provided. When bound to its receptor, IL18 signals through the MyD88/NF-κB signaling pathway. IL18 or IL18 receptor (IL18R) signaling is involved in tissue repair (e.g., in lungs and intestine) and reduction of inflammation. It is also involved in control of intestinal inflammation. Accordingly, in some aspects, provided herein is a chimeric signaling receptor for controlling IL18 or IL18R signaling.

In some embodiments, a chimeric signaling receptor for controlling IL18R signaling comprises a first polypeptide and a second polypeptide, wherein the first polypeptide comprises a first extracellular domain, a first transmembrane domain, and a first membrane proximal signaling domain, and the second polypeptide comprises a second extracellular domain, a second transmembrane domain, and a second membrane proximal signaling domain. In some embodiments, a first polypeptide further comprises a first membrane distal signaling domain, and a second polypeptide further comprises a second membrane distal signaling domain. IL18R domains may be comprised in either the membrane proximate signaling domains of the receptor polypeptides, or in the membrane distal signaling domains of the receptor polypeptides.

In some embodiments, a first extracellular domain comprises FKBP or a functional fragment thereof and a second extracellular domain comprises FRB or a functional fragment thereof. In some embodiments, a first membrane proximate domain of a chimeric signaling receptor for controlling IL18R signaling comprises an intracellular signaling domain or functional fragment of IL18R1, and a second membrane proximate domain of a chimeric signaling receptor for controlling IL18R signaling comprises an intracellular signaling domain or functional fragment of IL18RAP. In some embodiments, a first membrane proximate domain of a chimeric signaling receptor for controlling IL18R signaling comprises an intracellular signaling domain or functional fragment of IL18RAP, and a second membrane proximate domain of a chimeric signaling receptor for controlling IL18R signaling comprises an intracellular signaling domain or functional fragment of IL18R1.

In some embodiments, a chimeric signaling receptor for controlling Leukemia inhibitory factor (LIF) signaling is provided. LIF binds to the LIF receptor (LIFR) and recruits a gp130 to activate JAK/STAT3, PI3K/AKT, ERK1/2, and mTOR downstream signaling pathways. These pathways support immune tolerance, promote tissue repair, induce Foxp3 expression, and promote allo-tolerance. Accordingly, in some aspects, provided herein is a chimeric signaling receptor for controlling LIF or LIFR signaling.

In some embodiments a chimeric signaling receptor for controlling LIFR signaling comprises a first polypeptide and a second polypeptide, wherein the first polypeptide comprises a first extracellular domain, a first transmembrane domain, and a first membrane proximal signaling domain, and the second polypeptide comprises a second extracellular domain, a second transmembrane domain, and a second membrane proximal signaling domain. In some embodiments, a first polypeptide further comprises a first membrane distal signaling domain, and a second polypeptide further comprises a second membrane distal signaling domain. LIFR domains may be comprised in either the membrane proximate signaling domains of the receptor polypeptides, or in the membrane distal signaling domains of the receptor polypeptides.

In some embodiments, a first extracellular domain comprises FKBP or a functional fragment thereof and a second extracellular domain comprises FRB or a functional fragment thereof.

In some embodiments, a first membrane proximate domain of a chimeric signaling receptor for controlling LIFR signaling comprises an intracellular signaling domain or functional fragment of g130, and a second membrane proximate domain of a chimeric signaling receptor for controlling LIFR signaling comprises an intracellular signaling domain or functional fragment of LIFrβ. In some embodiments, a first membrane proximate domain of a chimeric signaling receptor for controlling LIFR signaling comprises an intracellular signaling domain or functional fragment of LIFrβ, and a second membrane proximate domain of a chimeric signaling receptor for controlling LIFR signaling comprises an intracellular signaling domain or functional fragment of gp130.

In some embodiments, a chimeric signaling receptor for controlling IL10 or IL10R signaling is provided. IL10 or IL10R signaling is involved in suppressive phenotype of Tr1 cells and has broad implications in tolerance induction in the context of inflammatory diseases such as irritable bowel disease (IBD) and Crohn's disease. Accordingly, in some aspects, provided herein is a chimeric signaling receptor for controlling IL10 or IL10R signaling.

In some embodiments a chimeric signaling receptor for controlling IL10R signaling comprises a first polypeptide and a second polypeptide, wherein the first polypeptide comprises a first extracellular domain, a first transmembrane domain, and a first membrane proximal signaling domain, and the second polypeptide comprises a second extracellular domain, a second transmembrane domain, and a second membrane proximal signaling domain. In some embodiments, a first polypeptide further comprises a first membrane distal signaling domain, and a second polypeptide further comprises a second membrane distal signaling domain. IL10R domains may be comprised in either the membrane proximate signaling domains of the receptor polypeptides, or in the membrane distal signaling domains of the receptor polypeptides.

In some embodiments, a first extracellular domain comprises FKBP or a functional fragment thereof and a second extracellular domain comprises FRB or a functional fragment thereof.

In some embodiments, a first membrane proximate domain of a chimeric signaling receptor for controlling IL10R signaling comprises an intracellular signaling domain or functional fragment of IL10R1, and a second membrane proximate domain of a chimeric signaling receptor for controlling IL10R signaling comprises an intracellular signaling domain or functional fragment of IL10R2. In some embodiments, a first membrane proximate domain of a chimeric signaling receptor for controlling IL10R signaling comprises an intracellular signaling domain or functional fragment of IL10R2, and a second membrane proximate domain of a chimeric signaling receptor for controlling IL10R signaling comprises an intracellular signaling domain or functional fragment of IL10R1.

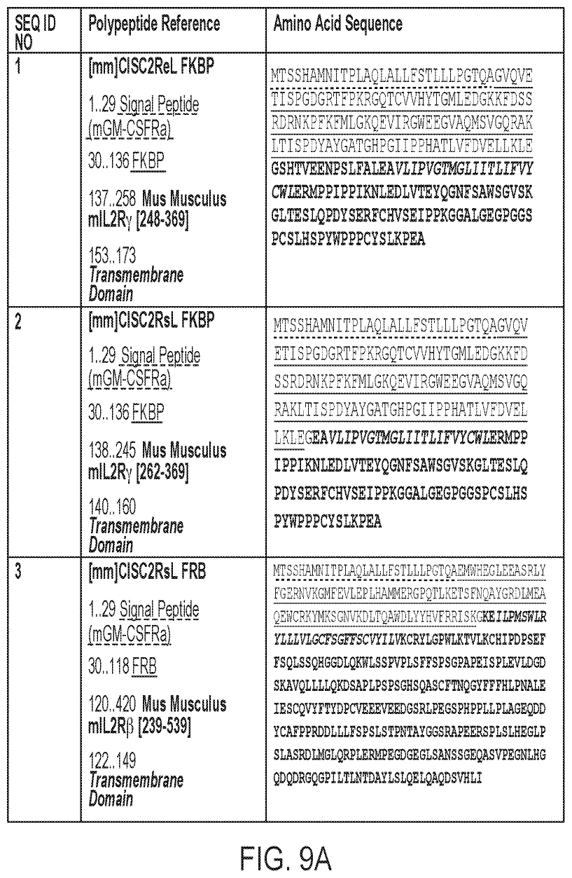

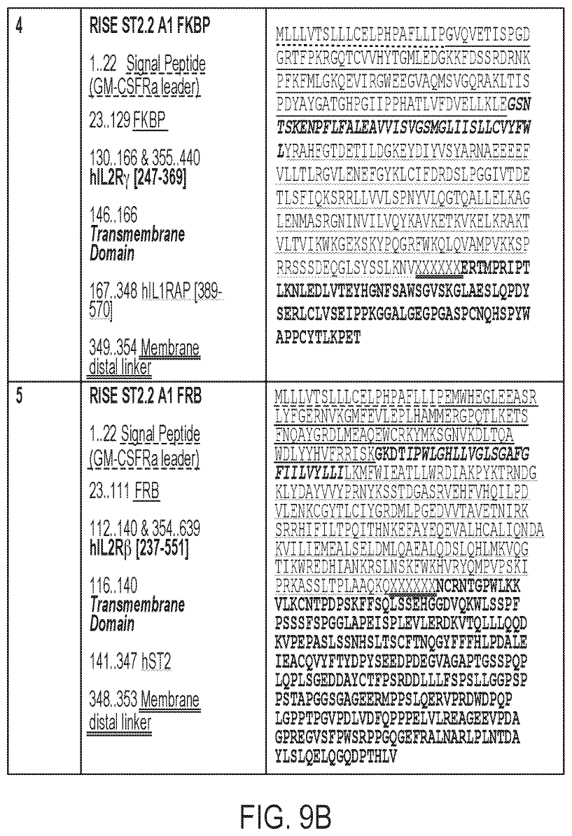

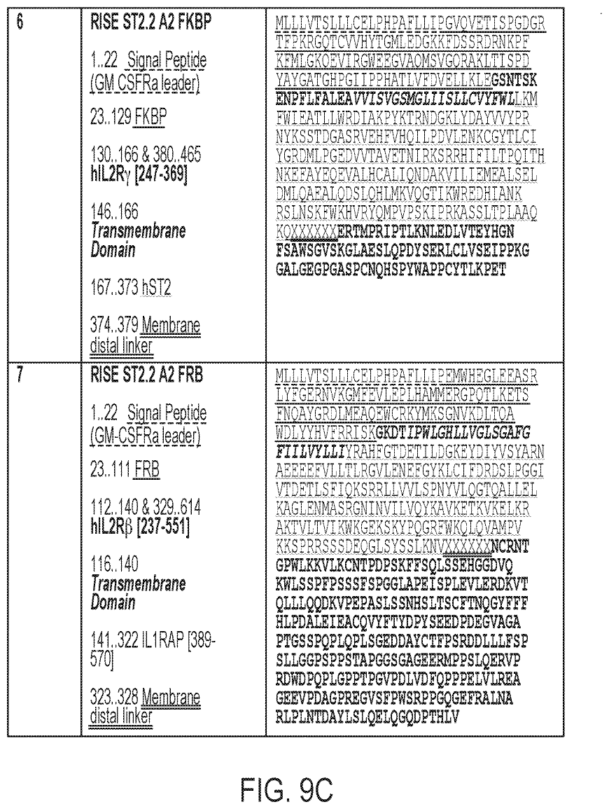

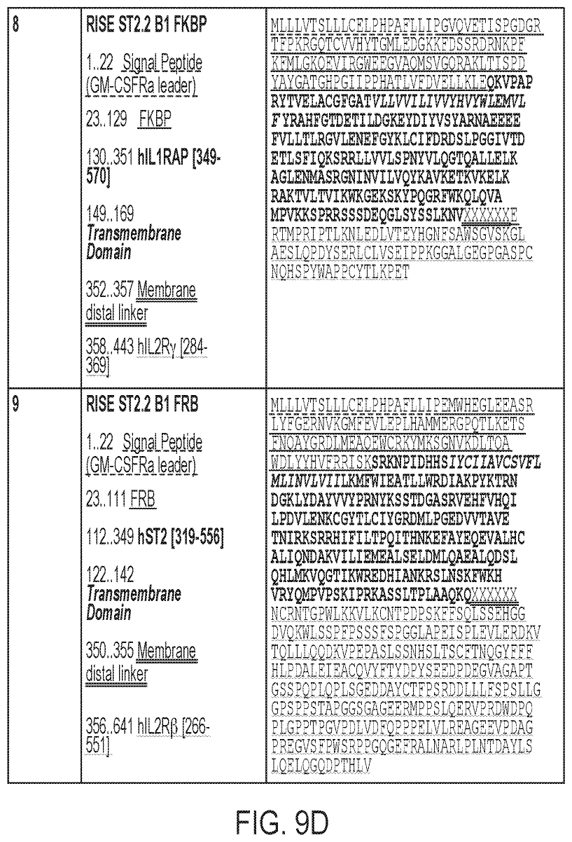

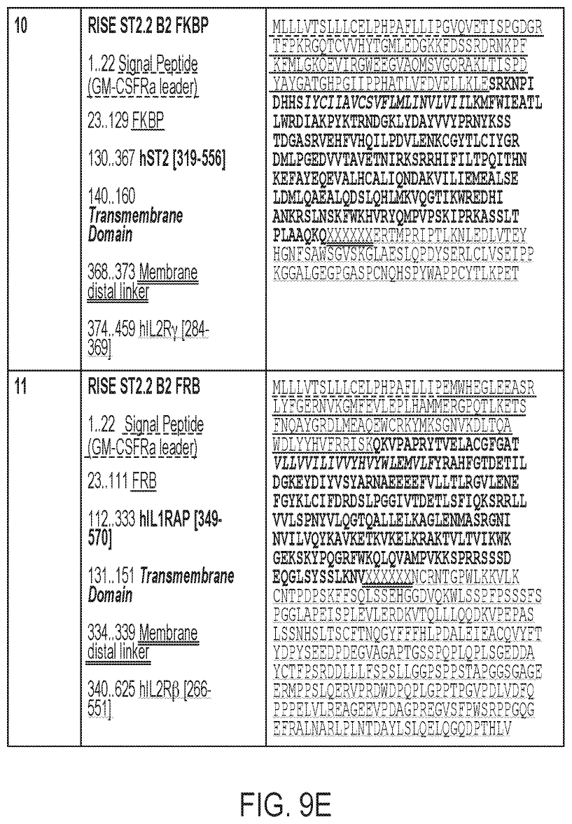

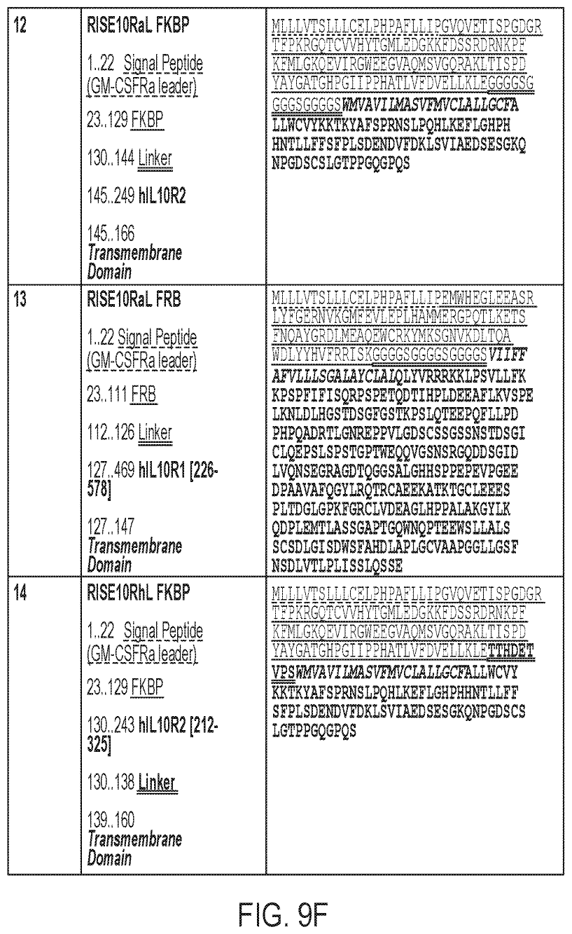

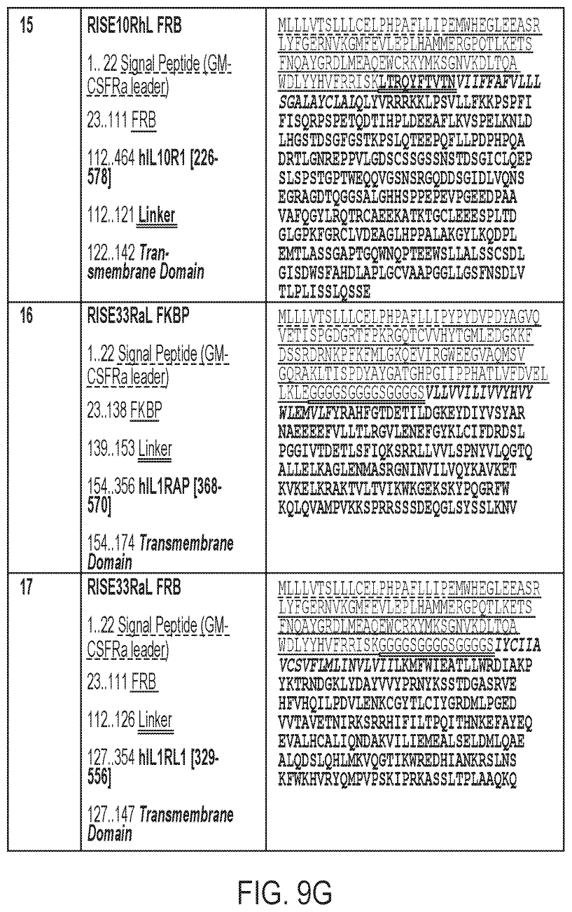

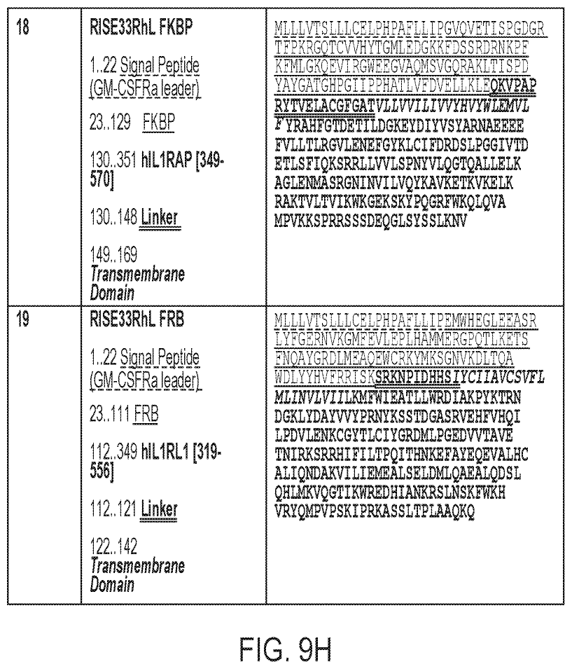

FIGS. 9A-9H provide examples of amino acid sequences of chimeric signaling receptors provided herein. A chimeric signaling receptor, or a domain thereof (e.g., a first polypeptide, a first extracellular domain, a first transmembrane domain, a first membrane proximal signaling domain, a first membrane distal signaling domain, a first extracellular linker, a first membrane proximal linker, a first membrane distal linker, a first signal peptide, a second polypeptide, a second extracellular domain, a second transmembrane domain, a second membrane proximal signaling domain, a second membrane distal signaling domain, a second extracellular linker, a second membrane proximal linker, a second membrane distal linker, or a second signal peptide) may comprise a polypeptide having at least 50% identity (e.g., at least 50, at least 60, at least 70, at least 80, at least 90, at least 95, at least 98, at least 99, or at least 99.5% identity) to any of the amino acid sequences provided in FIGS. 9A-9H.

### Compositions for Producing Engineered Cells

In some aspects, provided herein are compositions for engineering cells with any one of the chimeric signaling receptors described herein, either in vitro, in vivo, or ex vivo.

In some embodiments, provided herein are nucleic acids that comprise polynucleotides that encode any one of the chimeric signaling receptor polypeptides disclosed herein. In some embodiments, a nucleic acid as provided herein comprises a polynucleotide that encodes a first polypeptide of any one of the chimeric signaling receptors provided herein. In some embodiments, a nucleic acid as provided herein comprises a polynucleotide that encodes a second polypeptide of any one of the chimeric signaling receptors provided herein. In some embodiments, a nucleic acid as provided herein comprises a first polynucleotide that encodes a first polypeptide of any one of the chimeric signaling receptors provided herein and a second polynucleotide that encodes a second polypeptide of any one of the chimeric signaling receptors provided herein.

In some embodiments, nucleic acids comprising polynucleotides that encode any one of the chimeric signaling receptor polypeptides further comprise one or more control elements, e.g., a promoter that is operably linked to the one or more polynucleotides. Promoters can be constitutive or inducible.

In some embodiments, a composition for engineering cells according to the methods disclosed herein further comprises one or more nucleic acids encoding T cell receptors (TCRs), chimeric antigen receptors (CARs) to target particular cells, enhance suppressive function, or both. In some embodiments, a composition comprises a nucleic acid encoding IL-2. In some embodiments, a composition for making engineered cells according to the methods disclosed herein comprises a nucleic acid encoding a constitutively active IL-10 (see, e.g., WO 2019/180724, which is incorporated herein by reference in its entirety), CISC components, and/or a soluble FRB protein untethered from mTOR (see, e.g., WO 2018/111834, WO 2019/210057, and WO 2020/264039, each of which is incorporated herein by reference in its entirety).

In some embodiments, a nucleic acid described herein (e.g., for introduction into a cell or administration to a subject) is comprised in a vector. The term “vector” is used to refer to any molecule (e.g., nucleic acid, plasmid) or arrangement of molecules (e.g., virus) used to transfer coding information to a host cell. The term “expression vector” refers to a vector that is suitable for introduction of a host cell and contains nucleic acid sequences that direct and/or control expression of introduced heterologous nucleic acid sequences. Expression includes, but is not limited to, processes such as transcription, translation, and RNA splicing, if introns are present. Non-limiting examples of vectors include artificial chromosomes, minigenes, cosmids, plasmids, phagemids, and viral vectors. Non-limiting examples of viral vectors include lentiviral vectors, retroviral vectors, herpesvirus vectors, adenovirus vectors, and adeno-associated viral vectors. In some embodiments, one or more vectors comprising nucleic acids for use in the methods provided herein are lentiviral vectors. In some embodiments, one or more vectors are adenoviral vectors. In some embodiments, one or more vectors are adeno-associated viral (AAV) vectors. In some embodiments, one or more AAV vectors is an AAV1, AAV2, AAV3, AAV4, AAV5, AAV6, AAV7, AAV8, AAV9, AAV10, or AAV11 vector. In some embodiments, one or more AAV vectors are AAV5 vectors. In some embodiments, one or more AAV vectors are AAV6 vectors.

As will be understood by those skilled in the art, nucleic acids may include genomic sequences, extra-genomic and plasmid-encoded sequences and smaller engineered gene segments that express, or may be adapted to express, proteins, polypeptides, peptides and the like. Such segments may be naturally isolated or modified synthetically by the skilled person. As will be also recognized by the skilled artisan, polynucleotides may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide according to the present disclosure, and a polynucleotide may, but need not, be linked to other molecules and/or support materials. Polynucleotides may comprise a native sequence or may comprise a sequence encoding a variant or derivative of such a sequence.

In some embodiments, polynucleotide variants may have substantial identity to a reference polynucleotide sequence encoding an immunomodulatory polypeptide described herein. For example, a polynucleotide may be a polynucleotide comprising at least 70% sequence identity, preferably at least 75%, 80%, 85%, 90%, 95%, 96%, 97%, 98%, or 99% or higher, sequence identity or a sequence identity that is within a range defined by any two of the aforementioned percentages as compared to a reference polynucleotide sequence such as a sequence encoding an antibody described herein, using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

Typically, polynucleotide variants will contain one or more substitutions, additions, deletions and/or insertions, preferably such that the binding affinity of a polypeptide variant of a given polypeptide which is capable of a specific binding interaction with another molecule and is encoded by the variant polynucleotide is not substantially diminished relative to a polypeptide encoded by a polynucleotide sequence specifically set forth herein.

Some embodiments of nucleic acid sequences described herein (e.g., sequences on nucleic acids, vectors, or in cells) are codon-optimized for expression in a cell. The terms “codon-optimized” and “codon optimization,” with respect to a gene or coding sequence present in or introduced into a host cell, refer to alteration of codons in the gene or coding sequence to reflect the typical codon usage of the host cell, without altering the amino acid sequence encoded by the gene or coding sequence. Such optimization includes replacing at least one, or more than one, or a significant number, of codons with one or more codons that are more frequently used in the genes of that organism. Codon usage tables are readily available, for example, at the “Codon Usage Database” available at www.kazusa.or.jp. By utilizing the knowledge on codon usage or codon preference in each organism, one of ordinary skill in the art can apply the frequencies to any polypeptide with a given amino acid sequence, to produce a codon-optimized coding sequence which encodes the same polypeptide having the same amino acid sequence, but uses codons optimal for a given species (e.g., a human). Codon-optimized coding regions can be designed by various methods known to those skilled in the art.

The polynucleotides described herein, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of or about of 10,000, 5000, 3000, 2,000, 1,000, 500, 200, 100, or 50 base pairs in length, and the like, (including all intermediate lengths) are contemplated to be useful.

When comparing polynucleotide or nucleic acid sequences, two sequences are said to be “identical” if the sequence of nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least or at least about 20 contiguous positions, usually 30 to 75, or 40 to 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, WI), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J., Unified Approach to Alignment and Phylogenes, pp. 626-645 (1990); Methods in Enzymology vol. 183, Academic Press, Inc., San Diego, CA; Higgins, D. G. and Sharp, P. M., CABIOS 5:151-153 (1989); Myers, E. W. and Muller W., CABIOS 4:11-17 (1988); Robinson, E. D., Comb. Theor 11:105 (1971); Santou, N. Nes, M., Mol. Biol. Evol. 4:406-425 (1987); Sneath, P. H. A. and Sokal, R. R., Numerical Taxonomy—the Principles and Practice of Numerical Taxonomy, Freeman Press, San Francisco, CA (1973); Wilbur, W. J. and Lipman, D. J., Proc. Natl. Acad., Sci. USA 80:726-730 (1983).

Alternatively, optimal alignment of sequences for comparison may be conducted by the local identity algorithm of Smith and Waterman, Add. APL. Math 2:482 (1981), by the identity alignment algorithm of Needleman and Wunsch, J. Mol. Biol. 48:443 (1970), by the search for similarity methods of Pearson and Lipman, Proc. Natl. Acad. Sci. USA 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, WI), or by inspection.

One preferred example of algorithms that are suitable for determining percent sequence identity and sequence similarity are the BLAST and BLAST 2.0 algorithms, which are described in Altschul et al., Nucl Acids Res. 1977. 25:3389-3402, and Altschul et al., J Mol Biol. 1990. 215:403-410, respectively. BLAST and BLAST 2.0 can be used, for example with the parameters described herein, to determine percent sequence identity among two or more the polynucleotides. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff, Proc Natl Acad Sci USA. 1989. 89:10915) alignments, (B) of 50, expectation (E) of 10, M=5, N=−4 and a comparison of both strands.

In certain embodiments, the “percentage of sequence identity” is determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence (i.e., the window size) and multiplying the results by 100 to yield the percentage of sequence identity.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of patients.

### Cell Types

In some aspects, provided herein is a cell comprising any one of the nucleic acid molecules described herein, any one of the viral vectors described herein, or any one of the polypeptides described herein. In some aspects, provided herein is a cell comprising any one of the polypeptides of a chimeric signaling receptor provided herein, and/or one or more nucleic acids comprising polynucleotides encoding one or more polypeptides of a chimeric signaling receptor provided herein. In some embodiments, a cell as provided herein is a therapeutic cell, e.g., a cell that is prepared for administration to a subject (e.g., a human subject). A cell may be one of many cells cultured under certain conditions, or part of an organ that is harvested, part of an organoid, or an organism. In some embodiments, a cell disclosed herein is a eukaryotic cell (derived from a eukaryotic organism).

Embodiments of methods for producing genetically modified cells (e.g., by in vitro or ex vivo gene editing, and/or administration of compositions, vectors, or nucleic acids to a subject for in vivo editing) may use any cell type known in the art as a material for, e.g., introduction of nucleic acids, vectors, and/or compositions. It is to be understood that methods described herein that comprise manipulation of CD4+ cells, can be applied to other types of cells (e.g., CD25+, CD3+, CD8+, Foxp3+, and/or IL10/IL10R+ cells). In some embodiments, the methods described herein comprise editing an immune cell. Non-limiting examples of immune cells include B cells, T cells, and NK cells. In some embodiments, the methods provided herein comprise editing CD3+ cells, thereby producing edited CD3+ cells, including CD4+ and CD8+ Treg cells. In some embodiments, the methods comprise editing CD4+ T cells, thereby producing CD4+ Treg cells. In some embodiments, the methods comprise editing CD8+ T cells, thereby producing CD8+ Treg cells.

In some embodiments, the methods comprise editing a stem cell. In some embodiments, the methods comprise editing a pluripotent stem cell. In some embodiments, the methods comprise editing CD34+ hematopoietic stem cells (HSCs). In some embodiments, the methods comprise editing induced pluripotent stem cells (iPSCs). Edited stem cells may be matured in vitro to produce Treg cells, or administered to a subject to allow in vivo development into Treg cells. Edited stem cells may be matured into CD3+ Treg cells, CD4+ Treg cells, CD8+ Treg cells, or a combination thereof.

In some embodiments, a method comprises editing a T cell. A T cell or T lymphocyte is an immune system cell that matures in the thymus and produces a T cell receptor (TCR), e.g., an antigen-specific heterodimeric cell surface receptor typically comprised of an α-β heterodimer or a 7-6 heterodimer. T cells of a given clonality typically express only a single TCR clonotype that recognizes a specific antigenic epitope presented by a syngeneic antigen-presenting cell in the context of a major histocompatibility complex-encoded determinant. T cells can be naïve (“TN”; not exposed to antigen; increased expression of CD62L, CCR7, CD28, CD3, CD127, and CD45RA, and decreased or no expression of CD45RO as compared to TCM (described herein)), memory T cells (TM) (antigen experienced and long-lived), including stem cell memory T cells, and effector cells (antigen-experienced, cytotoxic). TM can be further divided into subsets of central memory T cells (TCM, expresses CD62L, CCR7, CD28, CD95, CD45RO, and CD127) and effector memory T cells (TEM, express CD45RO, decreased expression of CD62L, CCR7, CD28, and CD45RA). Effector T cells (TE) refers to antigen-experienced CD8+ cytotoxic T lymphocytes that express CD45RA, have decreased expression of CD62L, CCR7, and CD28 as compared to TCM, and are positive for granzyme and perforin. Helper T cells (TH) are CD4+ cells that influence the activity of other immune cells by releasing cytokines. CD4+ T cells can activate and suppress an adaptive immune response, and which of those two functions is induced will depend on the presence of other cells and signals. T cells can be collected using known techniques, and the various subpopulations or combinations thereof can be enriched or depleted by known techniques, for example, using antibodies that specifically recognize one or more T cell surface phenotypic markers, by affinity binding to antibodies, flow cytometry, fluorescence activated cell sorting (FACS), or immunomagnetic bead selection. Other exemplary T cells include regulatory T cells (Treg, also known as suppressor T cells), such as CD4+CD25+(FoxP3+) regulatory T cells and Treg17 cells, as well as Tr1, Th3, CD8+CD28−, or Qa-1 restricted T cells. In some embodiments, the cell is a CD3+, CD4+, and/or CD8+ T cell. In some embodiments, the cell is a CD3+ T cell. In some embodiments, the cell is a CD4+CD8-T cell. In some embodiments, the cell is a CD4-CD8+ T cell. In some embodiments, the cell is a regulatory T cell (Treg). Non-limiting examples of Treg cells are Tr1, Th3, CD8+CD28−, and Qa-1 restricted T cells. In some embodiments, the Treg cell is a FoxP3+ Treg cell. In some embodiments, the Treg cell expresses CTLA-4, LAG-3, CD25, CD39, CD27, CD70, CD357 (GITR), neuropilin-1, galectin-1, and/or IL-2Rα on its surface.

In some embodiments, the cell is a human cell. In some embodiments, a cell as described herein is isolated from a biological sample. A biological sample may be a sample from a subject (e.g., a human subject) or a composition produced in a lab (e.g., a culture of cells). A biological sample obtained from a subject make be a liquid sample (e.g., blood or a fraction thereof, a bronchial lavage, cerebrospinal fluid, or urine), or a solid sample (e.g., a piece of tissue) In some embodiments, the cell is obtained from peripheral blood. In some embodiments, the cell is obtained from umbilical cord blood. In some embodiments, the cell is obtained by sorting cells of peripheral blood to obtain a desired cell population (e.g., CD3+ cells), and one or more cells of the sorted population are modified by a method described herein. Also contemplated herein are cells produced by a method described herein.

Embodiments of genetically modified cells described herein may be any cell type known in the art. In some embodiments, the cell is a T cell, a precursor T cell, or a hematopoietic stem cell. In some embodiments, the cell is an NK-T cell (e.g., a FoxP3− NK-T cell or a FoxP3+NK-T cell). In some embodiments, the cell is a regulatory B (Breg) cell (e.g., a FoxP3− B cell or a FoxP3+B cell). In some embodiments, the cell is a CD4+ T cell (e.g., a FoxP3−CD4+ T cell or a FoxP3+CD4+ T cell) or a CD8+ T cell (e.g., a FoxP3−CD8+ T cell or a FoxP3+CD8+ T cell). In some embodiments, the cell is a CD25− T cell. In some embodiments, the cell is a regulatory T (Treg) cell. Non-limiting examples of Treg cells are Tr1, Th3, CD8+CD28−, and Qa-1 restricted T cells. In some embodiments, the Treg cell is a FoxP3+ Treg cell. In some embodiments, the Treg cell expresses CTLA-4, LAG-3, CD25, CD39, CD27, CD70, CD357 (GITR), neuropilin-1, galectin-1, and/or IL-2Rα on its surface. In some embodiments, a cell comprising or being engineered to comprise any one of the cell signaling receptors as provided herein are further engineered to stably express or overexpress FoxP3 (as described in WO18080541), membrane-bound IL10 (e.g., as described in WO2019180724), or a CAR or TCR. WO18080541 and WO2019180724 are incorporated herein by reference in their entirety.

In some embodiments, a cell is an autologous cell. In some embodiments, a cell is an allogeneic cell.

In some embodiments, a cell comprising a chimeric signaling receptor or nucleic acid encoding a chimeric signaling receptor further comprises or expresses a TCR or CAR.

### Stabilized FoxP3 Expression

Some embodiments of methods of modifying cells described herein comprise introducing a genetic modification in a cell that stabilizes expression of FoxP3. Similarly, some embodiments of cells described herein comprise a genetic modification that stabilizes or increases FoxP3 expression, relative to an unmodified cell. Additionally, some embodiments of nucleic acids and vectors described herein stabilize FoxP3 expression in a cell.

In some embodiments, an endogenous FOXP3 locus is modified in a cell, resulting in stabilized expression. For example, in some embodiments, a heterologous promoter is inserted within or downstream from a Treg-specific demethylated region (TSDR) in the genome, and upstream from a first coding exon of an endogenous FOXP3 coding sequence.

In some embodiments, a promoter is inserted downstream from the TSDR, and within or upstream from the first coding exon of FOXP3. Insertion of a heterologous promoter in this manner bypasses endogenous regulation of FOXP3 by the TSDR, which can become methylated in inflammatory conditions, inhibiting transcription of the endogenous FOXP3 coding sequence from the endogenous FOXP3 promoter located upstream from the TSDR. Thus, such stabilized FoxP3 expression by heterologous promoter insertion allows stable FoxP3 expression even in inflammatory conditions, preventing transdifferentiation into a T effector cell.

The heterologous promoter may be inserted at any position between the endogenous promoter and the first coding exon of the FOXP3 coding sequence. In some embodiments, the heterologous promoter is inserted 1-10,000, 10-1,000, 10-100, 10-5,000, 20-4,000, 30-3,000, 40-2,000, 50-1,000, 60-750, 70-500, 80-400, 90-300, 100-200, 1-1,000, 1,000-2,000, 2,000-3,000, 3,000-4,000, 4,000-5,000, 5,000-6,000, 6,000-7,000, 7,000-8,000, 8,000-9,000, or 9,000-10,000 nucleotides downstream from the TSDR of FOXP3. In some embodiments, the heterologous promoter is inserted 1-10,000, 10-1,000, 10-100, 10-5,000, 20-4,000, 30-3,000, 40-2,000, 50-1,000, 60-750, 70-500, 80-400, 90-300, 100-200, 1-1,000, 1,000-2,000, 2,000-3,000, 3,000-4,000, 4,000-5,000, 5,000-6,000, 6,000-7,000, 7,000-8,000, 8,000-9,000, or 9,000-10,000 nucleotides upstream from the first coding exon of the FOXP3 coding sequence. In some embodiments, the heterologous promoter is inserted into the first coding exon, such that a synthetic first coding exon is created, where the synthetic first coding exon differs from the endogenous first coding exon but still comprises a start codon that is in-frame with the FOXP3 coding sequence of downstream FOXP3 exons. In some embodiments, the heterologous promoter is inserted into the TSDR, such that the TSDR is modified and does not inhibit transcription of the endogenous FOXP3 coding sequence in inflammatory conditions.

In some embodiments, the nucleic acid comprising a heterologous promoter is comprised on a vector. In some embodiments, the vector is a viral vector. In some embodiments, the viral vector is an adeno-associated virus (AAV) vector. In some embodiments, the AAV vector is an AAV1, AAV2, AAV3, AAV4, AAV5, AAV6, AAV7, AAV8, AAV9, AAV10, or AAV11 vector. In some embodiments, the AAV vector is an AAV5 vector. In some embodiments, the AAV vector is an AAV6 vector.

In some embodiments, a nucleic acid comprising a promoter operably linked to a nucleic acid sequence encoding FoxP3 or a functional derivative thereof is introduced into the cell. Expression of a heterologous promoter and sequence encoding FoxP3 is useful, for example, for expressing functional FoxP3 in cells containing genomic mutations in the FOXP3 coding sequence (e.g., cells from subjects having IPEX syndrome). Additionally, additional coding sequences (e.g., encoding a chimeric signaling receptor described herein) may be included in a nucleic acid, such that the heterologous promoter controls transcription of RNA encoding FoxP3 sequence and one or more other proteins (e.g., a chimeric signaling receptor). In some embodiments, the sequence encoding FoxP3 is a cDNA sequence that does not comprise an intron.

The introduced nucleic acid may be integrated into the genome at a targeted locus (e.g., by homologous recombination), integrated in a non-targeted manner (e.g., by delivery on a lentiviral vector), or not integrated. In some embodiments, the nucleic acid comprises a 5′ homology arm that is upstream from the promoter, and a 3′ homology arm that is downstream from the nucleic acid sequence encoding FoxP3, and both homology arms have homology to a targeted locus in a genome. Such homology arms promote insertion of the nucleic acid into the genome at the targeted locus by homologous recombination. The homology arms may be the same length, have similar lengths (within 100 bp of each other), or different lengths. In some embodiments, one or both homology arms have a length of 200-2,000 bp, 400-1,500 bp, 500-1,000 bp. In some embodiments, one or both homology arms are about 100 bp, about 200 bp, about 300 bp, about 400 bp, about 500 bp, about 600 bp, about 700 bp, about 800 bp, about 900 bp, about 1,000 bp, about 1,100 bp, about 1,200 bp, about 1,300 bp, about 1,400 bp, about 1,500 bp, about 1,600 bp, about 1,700 bp, about 1,800 bp, about 1,900 bp, or about 2,000 bp.

In some embodiments, the nucleic acid is integrated at a FOXP3 locus in the genome. In some embodiments, the nucleic acid is integrated at a non-FOXP3 locus. In some embodiments, the targeted locus is a safe harbor locus. In some embodiments, the safe harbor locus is an AAVS1 locus, a HIPP11 locus, or a ROSA26 locus. In some embodiments, the nucleic acid is integrated at a TCRα (TRAC) locus. In some embodiments, the nucleic acid is integrated at a TCRβ (TRBC) locus.

In some embodiments, a nuclease capable of cleaving the genome at a targeted locus, or a nucleic acid encoding the nuclease (e.g., an mRNA) is introduced into the cell. Following delivery of the nuclease or transcription of the nuclease inside the cell, the nuclease introduces a double-stranded break at the targeted locus, thereby promoting integration of a donor template (e.g., nucleic acid comprising a promoter and sequence encoding FoxP3, or nucleic acid comprising a heterologous promoter for promoting transcription of an endogenous FOXP3 coding sequence) into the genome at the targeted locus by homology-directed repair. The nuclease may be any nuclease known in the art, including a meganuclease, zinc finger nuclease, TALEN, or RNA-guided nuclease. In embodiments where an RNA-guided nuclease (or nucleic acid encoding an RNA-guided nuclease) is delivered, a guide RNA (or nucleic acid encoding a guide RNA) comprising a spacer sequence complementary to a genomic sequence at the targeted locus is introduced into the cell. A gRNA or nucleic acid encoding a gRNA may be introduced into the cell with the nuclease or nucleic acid encoding the nuclease, or introduced separately (e.g., in a separate vector or delivery vehicle). The RNA-guided nuclease may be any RNA-guided nuclease known in the art or described herein.

In some embodiments, a nucleic acid comprising a heterologous promoter operably linked to a sequence encoding FoxP3 or a functional derivative thereof is present on a vector. In some embodiments, the vector is a viral vector. In some embodiments, the vector is a lentiviral vector. In some embodiments, the vector is an adeno-associated virus (AAV) vector. In some embodiments, the AAV vector is an AAV1, AAV2, AAV3, AAV4, AAV5, AAV6, AAV7, AAV8, AAV9, AAV10, or AAV11 vector. In some embodiments, the AAV vector is an AAV5 vector. In some embodiments, the AAV vector is an AAV6 vector. In some embodiments, the vector is a plasmid. In some embodiments, the vector is bacterial artificial chromosome. In some embodiments, the vector is human artificial chromosome. In some embodiments, the vector integrates into a chromosome of the genome, and RNA encoding FoxP3 is transcribed from the genome of the cell. In other embodiments, the vector does not integrate into a chromosome, and the sequence encoding FoxP3 is expressed episomally.

The heterologous promoter inserted into the FOXP3 locus or operably linked to the FOXP3 coding sequence may be any promoter known in the art. In some embodiments, the promoter is a constitutive promoter. In some embodiments, the promoter is an MND, PGK, or EF-1α promoter. In some embodiments, the promoter is an MND promoter. In some embodiments, the promoter is an inducible promoter. In some embodiments, the promoter is inducible by a drug or steroid.

### Antigen-Specific Receptor Polypeptides

Some embodiments of methods for producing engineered cells described herein include introducing a nucleic acid encoding an antigen-specific receptor (e.g., T cell receptor (TCR) or chimeric antigen receptor (CAR)) polypeptide, or portion thereof (e.g., a TCRα chain or TCRβ chain), into a cell. Similarly, some embodiments of cells described herein comprise a nucleic acid encoding an antigen-specific receptor. Additionally, some embodiments of nucleic acids and vectors described herein (e.g., for in vivo administration) encode a TCR or CAR or portion thereof. In some embodiments, a nucleic acid is inserted into, or comprises homology arms for directing insertion into, a TRAC locus or TRBC locus.

In some embodiments, a nucleic acid is inserted into or designed for insertion into the TRAC or TRBC locus to capture the endogenous promoter. Promoter capture includes the introduction of an exogenous sequence into a locus such that its expression is driven by the endogenous promoter. For example, a cell may be edited (ex vivo or in vivo) by inserting a nucleic acid molecule comprising a nucleic acid encoding an exogenous TCR or CAR or portion thereof into the TRAC or TRBC locus, where the nucleic acid encoding the TCR or CAR is inserted downstream of (e.g., 11 to 10,000 bp downstream from) the endogenous TRAC or TRBC promoter, such that the endogenous TRAC or TRBC promoter becomes operably linked to the inserted nucleic acid and drives expression of the exogenous TCR or CAR.

In some embodiments, a nucleic acid is inserted into or designed for insertion into a TRAC or TRBC locus, such that insertion disrupts expression of the endogenous TCRα or TCRβ chain. In some embodiments, the coding sequence of the endogenous TCRα or TCRβ chain, or a portion of the coding sequence, is removed from the locus such that the endogenous TCRα or TCRβ is not expressed in the cell. In some embodiments, the inserted nucleic acid comprises a heterologous promoter that drives expression of the inserted TCR or CAR.

In some embodiments, a nucleic acid is inserted into or designed for insertion into a TRAC or TRBC locus to hijack the endogenous TRAC or TRBC gene with a heterologous promoter. For example, a cell may be edited by inserting a polynucleotide molecule comprising a promoter operably linked to (a) a nucleic acid encoding a full-length TCRβ protein, and to a nucleic acid encoding TCRα variable (TRAV) and TCR joining (TRAJ) regions, where the coding sequences of the TRAV and TRAJ regions are inserted in-frame with the coding sequences encoding the TCRα constant regions, such that the inserted heterologous promoter controls transcription of a heterologous TCRβ protein and transcription of a TCRα protein comprising heterologous TRAV/TRAJ amino acid sequences and an endogenous TCRα constant region amino acid sequence. This embodiment utilizes the endogenous 3′ regulatory region from the endogenous TRAC gene. A similar approach may be used to hijack the endogenous TRBC locus, where the encoded full-length protein is a TCRα chain, and the nucleic acid further encodes TCRβ variable and TCRβ joining regions in-frame with TCRβ constant regions.

In some embodiments, an antigen-specific receptor is expressed episomally in a cell. Episomal expression may be achieved by any method known in the art, such as delivery of an RNA (e.g., mRNA or self-amplifying RNA) or DNA (e.g., plasmid or artificial chromosome) encoding the antigen-specific receptor.

In embodiments comprising use of a heterologous promoter to express a TCR, CAR, or portion thereof, the heterologous promoter may be any promoter known in the art. In some embodiments, the heterologous promoter is a constitutive promoter. In some embodiments, the promoter is an MND promoter.

### Pharmaceutical Compositions

Some aspects of the disclosure relate to a pharmaceutical composition comprising a cell, vector, or nucleic acid described herein, and a pharmaceutically acceptable excipient or carrier. Such pharmaceutical compositions are formulated, for example, for systemic administration, or administration to target tissues. “Acceptable” means that the excipient (carrier) must be compatible with the active ingredient of the composition (and preferably, capable of stabilizing the active ingredient) and not deleterious to the subject to be treated. Pharmaceutically acceptable excipients, carriers, buffers, stabilizers, isotonicizing agents, preservatives or antioxidants, or other materials well known to those skilled in the art. Such materials should be non-toxic and should not interfere with the efficacy of the active ingredient. The precise nature of the carrier or other material may depend on the route of administration, e.g., parenteral, intramuscular, intradermal, sublingual, buccal, ocular, intranasal, subcutaneous, intrathecal, intratumoral, oral, vaginal, or rectal. See, e.g., Remington: The Science and Practice of Pharmacy 20th Ed. (2000) Lippincott Williams and Wilkins, Ed. K. E. Hoover. The pharmaceutical compositions to be used for in vivo administration must be sterile, with the exception of any cells, viruses, and/or viral vectors being used to achieve a biological effect (e.g., immunosuppression). This is readily accomplished by, for example, filtration through sterile filtration membranes. The pharmaceutical compositions described herein may be placed into a container having a sterile access port, for example, an intravenous solution bag or vial having a stopper pierceable by a hypodermic injection needle.

In some embodiments, the pharmaceutical compositions described herein can be formulated for intramuscular injection, intravenous injection, intradermal injection, or subcutaneous injection.

The pharmaceutical compositions described herein to be used in the present methods can comprise pharmaceutically acceptable carriers, buffer agents, excipients, salts, or stabilizers in the form of lyophilized formulations or aqueous solutions. See, e.g., Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In some embodiments, the pharmaceutical composition described herein comprises lipid nanoparticles which can be prepared by methods known in the art, such as described in Epstein et al., Proc Natl Acad Sci USA 0.1985. 82:3688; Hwang et al. Proc Natl Acad Sci USA. 1980. 77:4030; and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Lipids used in the formulation of lipid nanoparticles for delivering nucleic acids are generally known in the art, and include ionizable amino lipids, non-cationic lipids, sterols, and polyethylene glycol-modified lipids. See, e.g., Buschmann et al., Vaccines. 2021. 9(1):65. In some embodiments, the nucleic acid is surrounded by the lipids of the lipid nanoparticle and present in the interior of the lipid nanoparticle. In some embodiments, the nucleic acid is dispersed throughout the lipids of the lipid nanoparticle. In some embodiments, the lipid nanoparticle comprises an ionizable amino lipid, a non-cationic lipid, a sterol, and/or a polyethylene glycol (PEG)-modified lipid.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device, or the nebulizing device may be attached to a face mask, tent or intermittent positive pressure breathing machine. Solution, suspension or powder compositions may be administered, preferably orally or nasally, from devices which deliver the formulation in an appropriate manner.

Pharmaceutical compositions described herein may be useful for treating a subject that has or is at risk of developing an inflammatory, autoimmune, or allergic condition or disease. A subject having or at risk of developing an inflammatory, autoimmune, or allergic condition or disease may be identified by ascertaining the presence and/or absence of one or more risk factors, diagnostic indicators, or prognostic indications. The determination may be made based on clinical, cellular, or serologic findings, including flow cytometry, serology, and/or DNA analyses known in the art.

The pharmaceutical compositions described herein can include a therapeutically effective amount of any cell, vector, and/or nucleic acid described herein. For example, in some embodiments, the pharmaceutical composition includes a cell, vector, or nucleic acid at any of the doses described herein.

A “therapeutically effective amount” refers to an amount effective, at dosages and for periods of time necessary, to achieve a desired therapeutic result. The therapeutically effective amount may vary according to factors such as the age, sex, and weight of the individual, and the ability of the cell, nucleic acid, or vector to effect a desired response in the subject.

Pharmaceutical compositions can be prepared in accordance with known techniques. See, e.g., Remington, The Science And Practice of Pharmacy (21st ed., Philadelphia, Lippincott, Williams & Wilkins, 2005). For example, cells, vectors, or nucleic acids described herein may be admixed with a pharmaceutically acceptable excipient, and the resulting composition is administered to a subject. The carrier must be acceptable in the sense of being compatible with any other ingredients in the formulation and must not be deleterious to the subject. The carrier can be a solid or a liquid, or both, and can be formulated with the compound as a unit-dose formulation.

In some embodiments, a pharmaceutical composition comprises cells at a dose of about 10⁴ to about 10¹⁰ cells/kg. In some embodiments, the pharmaceutical composition comprises cells at a dose of about: 10⁴ to 10⁵, 10⁵ to 10⁶, 10⁶ to 10⁷, 10⁷ to 10⁸, 10⁸ to 10⁹, or 10⁹ to 10¹⁰ cells/kg. In some embodiments, a pharmaceutical composition comprises cells at a dose of about 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2.0×10⁶, 2.1×10⁶, 2.2×10⁶, 2.3×10⁶, 2.4×10⁶, 2.5×10⁶, 2.6×10⁶, 2.7×10⁶, 2.8×10⁶, 2.9×10⁶, 3.0×10⁶, 3.1×10⁶, 3.2×10⁶, 3.3×10⁶, 3.4×10⁶, 3.5×10⁶, 3.6×10⁶, 3.7×10⁶, 3.8×10⁶, 3.9×10⁶, 4.0×10⁶, 4.1×10⁶, 4.2×10⁶, 4.3×10⁶, 4.4×10⁶, 4.5×10⁶, 4.6×10⁶, 4.7×10⁶, 4.8×10⁶, 4.9×10⁶, 5.0×10⁶, 5.1×10⁶, 5.2×10⁶, 5.3×10⁶, 5.4×10⁶, 5.5×10⁶, 5.6×10⁶, 5.7×10⁶, 5.8×10⁶, 5.9×10⁶, 6.0×10⁶, 6.1×10⁶, 6.2×10⁶, 6.3×10⁶, 6.4×10⁶, 6.5×10⁶, 6.6×10⁶, 6.7×10⁶, 6.8×10⁶, 6.9×10⁶, 7.0×10⁶, 7.1×10⁶, 7.2×10⁶, 7.3×10⁶, 7.4×10⁶, 7.5×10⁶, 7.6×10⁶, 7.7×10⁶, 7.8×10⁶, 7.9×10⁶, 8.0×10⁶, 8.1×10⁶, 8.2×10⁶, 8.3×10⁶, 8.4×10⁶, 8.5×10⁶, 8.6×10⁶, 8.7×10⁶, 8.8×10⁶, 8.9×10⁶, 9.0×10⁶, 9.1×10⁶, 9.2×10⁶, 9.3×10⁶, 9.4×10⁶, 9.5×10⁶, 9.6×10⁶, 9.7×10⁶, 9.8×10⁶, 9.9×10⁶, 1.0×10⁷, 1.1×10⁷, 1.2×10⁷, 1.3×10⁷, 1.4×10⁷, 1.5×10⁷, 1.6×10⁷, 1.7×10⁷, 1.8×10⁷, 1.9×10⁷, 2.0×10⁷, 2.1×10⁷, 2.2×10⁷, 2.3×10⁷, 2.4×10⁷, 2.5×10⁷, 2.6×10⁷, 2.7×10⁷, 2.8×10⁷, 2.9×10⁷, 3.0×10⁷, 3.1×10⁷, 3.2×10⁷, 3.3×10⁷, 3.4×10⁷, 3.5×10⁷, 3.6×10⁷, 3.7×10⁷, 3.8×10⁷, 3.9×10⁷, 4.0×10⁷, 4.1×10⁷, 4.2×10⁷, 4.3×10⁷, 4.4×10⁷, 4.5×10⁷, 4.6×10⁷, 4.7×10⁷, 4.8×10⁷, 4.9×10⁷, 5.0×10⁷, 5.1×10⁷, 5.2×10⁷, 5.3×10⁷, 5.4×10⁷, 5.5×10⁷, 5.6×10⁷, 5.7×10⁷, 5.8×10⁷, 5.9×10⁷, 6.0×10⁷, 6.1×10⁷, 6.2×10⁷, 6.3×10⁷, 6.4×10⁷, 6.5×10⁷, 6.6×10⁷, 6.7×10⁷, 6.8×10⁷, 6.9×10⁷, 7.0×10⁷, 7.1×10⁷, 7.2×10⁷, 7.3×10⁷, 7.4×10⁷, 7.5×10⁷, 7.6×10⁷, 7.7×10⁷, 7.8×10⁷, 7.9×10⁷, 8.0×10⁷, 8.1×10⁷, 8.2×10⁷, 8.3×10⁷, 8.4×10⁷, 8.5×10⁷, 8.6×10⁷, 8.7×10⁷, 8.8×10⁷, 8.9×10⁷, 9.0×10⁷, 9.1×10⁷, 9.2×10⁷, 9.3×10⁷, 9.4×10⁷, 9.5×10⁷, 9.6×10⁷, 9.7×10⁷, 9.8×10⁷, 9.9×10⁷, or 1.0×10⁸ cells/kg.

In some embodiments, a pharmaceutical composition comprises an effective amount of a vector or nucleic acid described herein. In some examples, the pharmaceutical composition comprises about 0.1 mg/kg to about 3 mg/kg of the vector or nucleic acid. In some embodiments, the pharmaceutical composition comprises about 0.1 mg/kg, about 0.25 mg/kg, about 0.5 mg/kg, about 0.75 mg/kg, about 1.0 mg/kg, about 1.5 mg/kg, about 2.0 mg/kg, about 2.5 mg/kg, or about 3.0 mg/kg of the vector or nucleic acid. In some embodiments, pharmaceutical composition comprises about 0.1 mg/kg to about 0.25 mg/kg, about 0.25 mg/kg to about 0.5 mg/kg, about 0.5 mg/kg to about 0.75 mg/kg, about 0.75 mg/kg to about 1.0 mg/kg, about 1.0 mg/kg to about 1.5 mg/kg, about 1.5 mg/kg to about 2.0 mg/kg, about 2.0 mg/kg to about 2.5 mg/kg, or about 2.5 mg/kg to about 3.0 mg/kg of the vector or nucleic acid.

In some embodiments, the pharmaceutical composition comprises a vector or nucleic acid encapsulated within a lipid nanoparticle.

Some embodiments of lipid nanoparticles comprise at least one cationic lipid, at least one non-cationic lipid, and at least one conjugated lipid. In more particular examples, lipid nanoparticles can comprise from about 50 mol % to about 85 mol % of a cationic lipid, from about 13 mol % to about 49.5 mol % of a non-cationic lipid, and from about 0.5 mol % to about 10 mol % of a lipid conjugate, and are produced in such a manner as to have a non-lamellar (i.e., non-bilayer) morphology. In other particular examples, lipid nanoparticles can comprise from about 40 mol % to about 85 mol % of a cationic lipid, from about 13 mol % to about 49.5 mol % of a non-cationic lipid, and from about 0.5 mol % to about 10 mol % of a lipid conjugate and are produced in such a manner as to have a non-lamellar (i.e., non-bilayer) morphology.

Cationic lipids can include, for example, one or more of the following: palmitoyi-oleoyl-nor-arginine (PONA), MPDACA, GUADACA, ((6Z,9Z,28Z,31Z)-heptatriaconta-6,9,28,31-tetraen-19-yl 4-(dimethylamino)butanoate) (MC3), LenMC3, CP-LenMC3, 7-LenMC3, CP-γ-LenMC3, MC3MC, MC2MC, MC3 Ether, MC4 Ether, MC3 Amide, Pan-MC3, Pan-MC4 and Pan MC5, 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-K-C2-DMA; “XTC2”), 2,2-dilinoleyl-4-(3-dimethylaminopropyl)-[1,3]-dioxolane (DLin-K-C3-DMA), 2,2-dilinoleyl-4-(4-dimethylaminobutyl)-[1,3]-dioxolane (DLin-K-C4-DMA), 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dilinoleyl-4-dimethylaminomethyl-[1,3]-dioxolane (DLin-K-DMA), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.C1), 1,2-dilinoleoyl-3-trimethylaminopropane chloride salt (DLin-TAP.C1), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), N,N-dioleyl-N,N-dimethylammonium chloride (DODAC), 1,2-dioleyloxy-N,N-dimethylaminopropane (DODMA), 1,2-distearyloxy-N,N-dimethylaminopropane (DSDMA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N—(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethy-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), or mixtures thereof. The cationic lipid can also be DLinDMA, DLin-K-C2-DMA (“XTC2”), MC3, LenMC3, CP-LenMC3, 7-LenMC3, CP-γ-LenMC3, MC3MC, MC2MC, MC3 Ether, MC4 Ether, MC3 Amide, Pan-MC3, Pan-MC4, Pan MC5, or mixtures thereof.

In various embodiments, the cationic lipid may comprise from about 50 mol % to about 90 mol %, from about 50 mol % to about 85 mol %, from about 50 mol % to about 80 mol %, from about 50 mol % to about 75 mol %, from about 50 mol % to about 70 mol %, from about 50 mol % to about 65 mol %, or from about 50 mol % to about 60 mol % of the total lipid present in the particle.

In other embodiments, the cationic lipid may comprise from about 40 mol % to about 90 mol %, from about 40 mol % to about 85 mol %, from about 40 mol % to about 80 mol % from about 40 mol % to about 75 mol %, from about 40 mol % to about 70 mol %, from about 40 mol % to about 65 mol %, or from about 40 mol % to about 60 mol % of the total lipid present in the particle.

The non-cationic lipid may comprise, e.g., one or more anionic lipids and/or neutral lipids. In particular embodiments, the non-cationic lipid comprises one of the following neutral lipid components: (1) cholesterol or a derivative thereof; (2) a phospholipid; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, and mixtures thereof. The phospholipid may be a neutral lipid including, but not limited to, dipalmitoylphosphatidylcholine (DPPC), distearoylphosphatidylcholine (DSPC), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), and mixtures thereof. In certain embodiments, the phospholipid is DPPC, DSPC, or mixtures thereof.

In some embodiments, the non-cationic lipid (e.g., one or more phospholipids and/or cholesterol) may comprise from about 10 mol % to about 60 mol %, from about 15 mol % to about 60 mol %, from about 20 mol % to about 60 mol %, from about 25 mol % to about 60 mol %, from about 30 mol % to about 60 mol %, from about 10 mol % to about 55 mol %, from about 15 mol % to about 55 mol %, from about 20 mol % to about 55 mol %, from about 25 mol % to about 55 mol %, from about 30 mol % to about 55 mol %, from about 13 mol % to about 50 mol %, from about 15 mol % to about 50 mol % or from about 20 mol % to about 50 mol % of the total lipid present in the particle. When the non-cationic lipid is a mixture of a phospholipid and cholesterol or a cholesterol derivative, the mixture may comprise up to about 40, 50, or 60 mol % of the total lipid present in the particle.

The conjugated lipid that inhibits aggregation of particles may comprise, e.g., one or more of the following: a polyethyleneglycol (PEG)-lipid conjugate, a polyamide (ATTA)-lipid conjugate, a cationic-polymer-lipid conjugates (CPLs), or mixtures thereof. In one particular embodiment, the nucleic acid-lipid particles comprise either a PEG-lipid conjugate or an ATTA-lipid conjugate. In certain embodiments, the PEG-lipid conjugate or ATTA-lipid conjugate is used together with a CPL. The conjugated lipid that inhibits aggregation of particles may comprise a PEG-lipid including, e.g., a PEG-diacylglycerol (DAG), a PEG dialkyloxypropyl (DAA), a PEG-phospholipid, a PEG-ceramide (Cer), or mixtures thereof. The PEG-DAA conjugate may be PEG-di lauryloxypropyl (C12), a PEG-dimyristyloxypropyl (C14), a PEG-dipalmityloxypropyl (C16), a PEG-distearyloxypropyl (C18), or mixtures thereof.

Additional PEG-lipid conjugates suitable for use in the invention include, but are not limited to, mPEG2000-1,2-di-O-alkyl-sn3-carbomoylglyceride (PEG-C-DOMG). The synthesis of PEG-C-DOMG is described in PCT Application No. PCT/US08/88676. Yet additional PEG-lipid conjugates suitable for use in the invention include, without limitation, 1-[8′-(1,2-dimyristoyl-3-propanoxy)-carboxamido-3′,6′-dioxaoctanyl]carbamoyl-ω-methyl-poly(ethylene glycol) (2KPEG-DMG). The synthesis of 2KPEG-DMG is described in U.S. Pat. No. 7,404,969.

In some cases, the conjugated lipid that inhibits aggregation of particles (e.g., PEG-lipid conjugate) may comprise from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 1 mol % to about 1.8 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1.4, 1.5, 1.6, 1.7, 1.8, 1.9, or 2 mol % (or any fraction thereof or range therein) of the total lipid present in the particle. Typically, in such instances, the PEG moiety has an average molecular weight of about 2,000 Daltons. In other cases, the conjugated lipid that inhibits aggregation of particles (e.g., PEG-lipid conjugate) may comprise from about 5.0 mol % to about 10 mol %, from about 5 mol % to about 9 mol %, from about 5 mol % to about 8 mol %, from about 6 mol % to about 9 mol %, from about 6 mol % to about 8 mol %, or about 5 mol %, 6 mol %, 7 mol %, 8 mol %, 9 mol %, or 10 mol % (or any fraction thereof or range therein) of the total lipid present in the particle. Typically, in such instances, the PEG moiety has an average molecular weight of about 750 Daltons.

In other embodiments, the composition may comprise amphoteric liposomes, which contain at least one positive and at least one negative charge carrier, which differs from the positive one, the isoelectric point of the liposomes being between 4 and 8. This objective is accomplished owing to the fact that liposomes are prepared with a pH-dependent, changing charge.

Liposomal structures with the desired properties are formed, for example, when the amount of membrane-forming or membrane-based cationic charge carriers exceeds that of the anionic charge carriers at a low pH and the ratio is reversed at a higher pH. This is always the case when the ionizable components have a pKa value between 4 and 9. As the pH of the medium drops, all cationic charge carriers are more charged and all anionic charge carriers lose their charge.

Cationic compounds useful for amphoteric liposomes include those cationic compounds previously described herein above. Without limitation, strongly cationic compounds can include, for example: DC-Chol 3-β-[N—(N′,N′-dimethylmethane) carbamoyl] cholesterol, TC-Chol 3-β-[N—(N′, N′, N′-trimethylaminoethane) carbamoyl cholesterol, BGSC bisguanidinium-spermidine-cholesterol, BGTC bis-guadinium-tren-cholesterol, DOTAP (1,2-dioleoyloxypropyl)-N,N,N-trimethylammonium chloride, DOSPER (1,3-dioleoyloxy-2-(6-carboxy-spermyl)-propylarnide, DOTMA (1,2-dioleoyloxypropyl)-N,N,N-trimethylamronium chloride) (Lipofectin®), DORIE 1,2-dioleoyloxypropyl)-3-dimethylhydroxyethylammonium bromide, DOSC (1,2-dioleoyl-3-succinyl-sn-glyceryl choline ester), DOGSDSO (1,2-dioleoyl-sn-glycero-3-succinyl-2-hydroxyethyl disulfide omithine), DDAB dimethyldioctadecylammonium bromide, DOGS ((C18)2GlySper3+) N,N-dioctadecylamido-glycol-spermin (Transfectam®) (C18)2Gly+N,N-dioctadecylamido-glycine, CTAB cetyltrimethylarnmonium bromide, CpyC cetylpyridinium chloride, DOEPC 1,2-dioleoly-sn-glycero-3-ethylphosphocholine or other O-alkyl-phosphatidylcholine or ethanolamines, amides from lysine, arginine or ornithine and phosphatidyl ethanolamine.

Examples of weakly cationic compounds include, without limitation: His-Chol (histaminyl-cholesterol hemisuccinate), Mo-Chol (morpholine-N-ethylamino-cholesterol hemisuccinate), or histidinyl-PE.

Examples of neutral compounds include, without limitation: cholesterol, ceramides, phosphatidyl cholines, phosphatidyl ethanolamines, tetraether lipids, or diacyl glycerols.

Anionic compounds useful for amphoteric liposomes include those non-cationic compounds previously described herein. Without limitation, examples of weakly anionic compounds can include: CHEMS (cholesterol hemisuccinate), alkyl carboxylic acids with 8 to 25 carbon atoms, or diacyl glycerol hemisuccinate. Additional weakly anionic compounds can include the amides of aspartic acid, or glutamic acid and PE as well as PS and its amides with glycine, alanine, glutamine, asparagine, serine, cysteine, threonine, tyrosine, glutamic acid, aspartic acid or other amino acids or aminodicarboxylic acids. According to the same principle, the esters of hydroxycarboxylic acids or hydroxydicarboxylic acids and PS are also weakly anionic compounds.

In some embodiments, amphoteric liposomes may contain a conjugated lipid, such as those described herein above. Particular examples of useful conjugated lipids include, without limitation, PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Particular examples are PEG-modified diacylglycerols and dialkylglycerols.

In some embodiments, the neutral lipids may comprise from about 10 mol % to about 60 mol %, from about 15 mol % to about 60 mol %, from about 20 mol % to about 60 mol %, from about 25 mol % to about 60 mol %, from about 30 mol % to about 60 mol %, from about 10 mol % to about 55 mol %, from about 15 mol % to about 55 mol %, from about 20 mol % to about 55 mol %, from about 25 mol % to about 55 mol %, from about 30 mol % to about 55 mol %, from about 13 mol % to about 50 mol %, from about 15 mol % to about 50 mol % or from about 20 mol % to about 50 mol % of the total lipid present in the particle.

In some cases, the conjugated lipid that inhibits aggregation of particles (e.g., PEG-lipid conjugate) may comprise from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 1 mol % to about 1.8 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1.4, 1.5, 1.6, 1.7, 1.8, 1.9, or 2 mol % (or any fraction thereof or range therein) of the total lipid present in the particle. Typically, in such instances, the PEG moiety has an average molecular weight of about 2,000 Daltons. In other cases, the conjugated lipid that inhibits aggregation of particles (e.g., PEG-lipid conjugate) may comprise from about 5.0 mol % to about 10 mol %, from about 5 mol % to about 9 mol %, from about 5 mol % to about 8 mol %, from about 6 mol % to about 9 mol %, from about 6 mol % to about 8 mol %, or about 5 mol %, 6 mol %, 7 mol %, 8 mol %, 9 mol %, or 10 mol % (or any fraction thereof or range therein) of the total lipid present in the particle. Typically, in such instances, the PEG moiety has an average molecular weight of about 750 Daltons.

Considering the total amount of neutral and conjugated lipids, the remaining balance of the amphoteric liposome can comprise a mixture of cationic compounds and anionic compounds formulated at various ratios. The ratio of cationic to anionic lipid may selected in order to achieve the desired properties of nucleic acid encapsulation, zeta potential, pKa, or other physicochemical property that is at least in part dependent on the presence of charged lipid components.

In some embodiments, the lipid nanoparticles have a composition that specifically enhances delivery and uptake in stem cells, hematopoietic cells, or T cells.

In some embodiments, the pharmaceutical composition comprises an effective amount of a lipid nanoparticle formulation, wherein the lipid nanoparticle formulation comprises a vector or nucleic acid described herein. In some examples, the lipid nanoparticle formulation comprises about 0.1 mg/kg to about 3 mg/kg of the vector or nucleic acid. In some embodiments, the lipid nanoparticle formulation comprises about 0.1 mg/kg, about 0.25 mg/kg, about 0.5 mg/kg, about 0.75 mg/kg, about 1.0 mg/kg, about 1.5 mg/kg, about 2.0 mg/kg, about 2.5 mg/kg, or about 3.0 mg/kg of the vector or nucleic acid. In some embodiments, the lipid nanoparticle formulation comprises about 0.1 mg/kg to about 0.25 mg/kg, about 0.25 mg/kg to about 0.5 mg/kg, about 0.5 mg/kg to about 0.75 mg/kg, about 0.75 mg/kg to about 1.0 mg/kg, about 1.0 mg/kg to about 1.5 mg/kg, about 1.5 mg/kg to about 2.0 mg/kg, about 2.0 mg/kg to about 2.5 mg/kg, or about 2.5 mg/kg to about 3.0 mg/kg of the vector or nucleic acid.

In some embodiments, the pharmaceutical composition comprises an effective amount of a lipid nanoparticle formulation comprising a donor template comprising a template nucleic acid described herein, wherein lipid nanoparticle formulation comprises about 0.1 mg/kg to about 3 mg/kg of the donor polynucleotide. In some embodiments, the lipid nanoparticle formulation comprises about 0.1 mg/kg, about 0.25 mg/kg, about 0.5 mg/kg, about 0.75 mg/kg, about 1.0 mg/kg, about 1.5 mg/kg, about 2.0 mg/kg, about 2.5 mg/kg, or about 3.0 mg/kg of the donor polynucleotide. In some embodiments, the lipid nanoparticle formulation comprises about 0.1 mg/kg to about 0.25 mg/kg, about 0.25 mg/kg to about 0.5 mg/kg, about 0.5 mg/kg to about 0.75 mg/kg, about 0.75 mg/kg to about 1.0 mg/kg, about 1.0 mg/kg to about 1.5 mg/kg, about 1.5 mg/kg to about 2.0 mg/kg, about 2.0 mg/kg to about 2.5 mg/kg, or about 2.5 mg/kg to about 3.0 mg/kg of the donor polynucleotide.

In some embodiments, pharmaceutical compositions of the invention can further comprise one or more additional agents useful in the treatment of an inflammatory, autoimmune, or allergic condition or disease in a subject.

### Methods of Use

Some aspects of the disclosure relate to methods of administering a cell, vector, nucleic acid, or lipid nanoparticle described herein to a subject. In some embodiments, a method comprises administering to a subject any one of the cells described herein. In some embodiments, a method comprises administering to the subject a cell that had previously been obtained from that subject before being administered (i.e., the cell is an autologous cell).

In some embodiments, a method comprises (i) isolation of cells from a subject; (ii) processing the cells by any method (e.g., gene editing or introducing a vector) described herein; and (iii) administering the processed cells to the same subject. In some embodiments, a method comprises administering to the subject a cell that had previously been obtained from a different subject than the one to whom the cell is administered (i.e., the cell is an allogeneic cell). In some embodiments, a method comprises (i) isolation of cells from a first subject; (ii) processing the cells by any method (e.g., gene editing or introducing a vector) described herein; and (iii) administering the processed cells to a second subject.

Some embodiments of the methods, cells, systems, and compositions described herein include any of the cells, vectors, nucleic acids, or lipid nanoparticles described herein, for use as a medicament. In some embodiments, the cell, vector, nucleic acid, or lipid nanoparticle is for use in a method of preventing, treating, inhibiting, or ameliorating an inflammatory, autoimmune, or allergic condition or disease in a subject.

In some embodiments, a cell is described herein for use in a method of preventing, treating, inhibiting, or ameliorating an inflammatory, autoimmune, or allergic condition or disease in a subject. In some embodiments, the cell is autologous to the subject (i.e., derived from the subject). In other embodiments, the cell is allogeneic to the subject (i.e., derived from a different subject).

### Inflammatory Diseases

In some embodiments, a cell, vector, nucleic acid, or lipid nanoparticle is used for treating, preventing, treating, inhibiting, or ameliorating an inflammatory condition or disease in a subject. In some embodiments, the subject has or is at risk of developing an inflammatory condition or disease. In some embodiments, the inflammatory condition or disease is selected from pancreatic islet cell transplantation, asthma, hepatitis, traumatic brain injury, primary sclerosing cholangitis, primary biliary cholangitis, polymyositis, stroke, Still's disease, acute respiratory distress syndrome (ARDS), uveitis, inflammatory bowel disease (IBD), ulcerative colitis, graft-versus-host disease (GvHD), tolerance induction for transplantation, transplant rejection, and sepsis.

In some embodiments, the inflammatory condition is associated with pancreatic islet cell transplantation. In some embodiments, the inflammatory disease is asthma. In some embodiments, the inflammatory disease is hepatitis. In some embodiments, the inflammatory condition is traumatic brain injury. In some embodiments, the inflammatory disease is primary sclerosing cholangitis. In some embodiments, the inflammatory disease is primary biliary cholangitis. In some embodiments, the inflammatory disease is polymyositis. In some embodiments, the inflammatory condition is stroke. In some embodiments, the inflammatory disease is Still's disease. In some embodiments, the inflammatory disease is acute respiratory distress syndrome (ARDS). In some embodiments, the inflammatory disease is uveitis. In some embodiments, the inflammatory disease is inflammatory bowel disease (IBD). In some embodiments, the inflammatory disease is graft-versus-host disease (GvHD). In some embodiments, the inflammatory condition is tolerance induction for transplantation. In some embodiments, the inflammatory condition is transplant rejection. In some embodiments, the inflammatory disease is sepsis.

In some embodiments, the cell expresses an antigen-specific receptor (e.g., T cell receptor or chimeric antigen receptor) that is specific to an antigen associated with the inflammatory condition or disease.

### Autoimmune Diseases

In some embodiments, a cell, vector, nucleic acid, or lipid nanoparticle is used for treating, preventing, treating, inhibiting, or ameliorating an autoimmune condition or disease in a subject. In some embodiments, the subject has or is at risk of developing an autoimmune condition or disease. In some embodiments, the autoimmune condition or disease is selected from type 1 diabetes mellitus, multiple sclerosis, systemic lupus erythematosus, myasthenia gravis, rheumatoid arthritis, early onset rheumatoid arthritis, ankylosing spondylitis, immune-mediated pregnancy loss, immune-mediated recurrent pregnancy loss, dermatomyositis, psoriatic arthritis, Crohn's disease, inflammatory bowel disease (IBD), ulcerative colitis, bullous pemphigoid, pemphigus vulgaris, autoimmune hepatitis, psoriasis, Sjogren's syndrome, and celiac disease.

In some embodiments, the autoimmune disease is type 1 diabetes mellitus. In some embodiments, the autoimmune disease is multiple sclerosis. In some embodiments, the autoimmune disease is systemic lupus erythematosus. In some embodiments, the autoimmune disease is myasthenia gravis. In some embodiments, the autoimmune disease is rheumatoid arthritis. In some embodiments, the autoimmune disease is early onset rheumatoid arthritis. In some embodiments, the autoimmune disease is ankylosing spondylitis. In some embodiments, the autoimmune disease is immune-mediated pregnancy loss. In some embodiments, the autoimmune disease is immune-mediated recurrent pregnancy loss. In some embodiments, the autoimmune disease is dermatomyositis. In some embodiments, the autoimmune disease is psoriatic arthritis. In some embodiments, the autoimmune disease is Crohn's disease. In some embodiments, the autoimmune disease is inflammatory bowel disease (IBD). In some embodiments, the autoimmune disease is ulcerative colitis. In some embodiments, the autoimmune disease is bullous pemphigoid. In some embodiments, the autoimmune disease is pemphigus vulgaris. In some embodiments, the autoimmune disease is autoimmune hepatitis. In some embodiments, the autoimmune disease is psoriasis. In some embodiments, the autoimmune disease is Sjogren's syndrome. In some embodiments, the autoimmune disease is celiac disease.

In some embodiments, the cell expresses an antigen-specific receptor (e.g., T cell receptor or chimeric antigen receptor) that is specific to an antigen associated with the autoimmune disease.

### Allergic Diseases

In some embodiments, a cell, vector, nucleic acid, or lipid nanoparticle is used for treating, preventing, treating, inhibiting, or ameliorating an allergic condition or disease in a subject. In some embodiments, the subject has or is at risk of developing an allergic condition or disease. In some embodiments, the allergic condition or disease is selected from allergic asthma, steroid-resistant asthma, atopic dermatitis, celiac disease, pollen allergy, food allergy, drug hypersensitivity, and contact dermatitis. In some embodiments, the allergic disease is allergic asthma. In some embodiments, the allergic disease is steroid-resistant asthma. In some embodiments, the allergic disease is atopic dermatitis. In some embodiments, the allergic disease is celiac disease. In some embodiments, the allergic disease is pollen allergy. In some embodiments, the allergic disease is food allergy. In some embodiments, the allergic disease is drug hypersensitivity. In some embodiments, the allergic disease is contact dermatitis.

In some embodiments, the cell expresses an antigen-specific receptor (e.g., T cell receptor or chimeric antigen receptor) that is specific to an antigen associated with the allergic disease.

### Administration

In some embodiments, a cell, vector, nucleic acid, or lipid nanoparticle may be administered between 1 and 14 days over a 30-day period. In some embodiments, doses may be provided 1, 2, 3, 4, 5, 6, 7, 8, 9, 10, 11, 12, 13, or 14 days over a 60-day period. Alternate protocols may be appropriate for individual subjects. A suitable dose is an amount of a compound that, when administered as described above, is capable of detectably altering or ameliorating symptoms, or decreases at least one indicator of autoimmune, allergic or other inflammatory immune activity in a statistically significant manner by at least 10-50% relative to the basal (e.g., untreated) level, which can be monitored by measuring specific levels of blood components, for example, detectable levels of circulating immunocytes and/or other inflammatory cells and/or soluble inflammatory mediators including proinflammatory cytokines.

In some embodiments, rapamycin or a rapalog is administered to the subject before the administration of cells, in conjunction with cells, and/or following the administration of cells. Administration of rapamycin or a rapalog that is capable of inducing dimerization of the CISC components on the surface of a cell results in continued IL2 signal transduction in vivo, promoting survival and proliferation of the CISC-expressing cell without the undesired effects that would be caused by IL2 administration, such as activation of other T cells. In some embodiments, the rapamycin or rapalog that is administered is everolimus, CCI-779, C20-methallylrapamycin, C16-(S)-3-methylindolerapamycin, C16-iRap, C16-(S)-7-methylindolerapamycin, AP21967, C16-(S)Butylsulfonamidorapamycin, AP23050, sodium mycophenolic acid, benidipine hydrochloride, AP1903, and AP23573, or a metabolite or derivative thereof. In some embodiments, the rapamycin or rapalog is administered at a dose of 0.001 mg/kg to 10 mg/kg body mass of the subject, or a dose between 0.001 mg/kg and 10 mg/kg. In some embodiments, the rapamycin or rapalog is administered at a dose of 0.001 mg/kg to 0.01 mg/kg, 0.01 mg/kg to 0.1 mg/kg, 0.1 mg/kg to 1 mg/kg, or 1 mg/kg to 10 mg/kg. In some embodiments, the rapamycin or rapalog is administered in a separate composition from the cells. In some embodiments, the rapamycin or rapalog is administered in multiple doses. In some embodiments, the rapamycin or rapalog is administered for 1, 2, 3, 4, 5, 6, 7, 8, 9, 10, 11, 12, 13, or 14 or more days. In some embodiments, the rapamycin or rapalog is administered for 1, 2, 3, 4, 5, 6, 7, 8, 9, 10, 11, 12, 13, 14, 15, 16, 17, 18, 19, or 20 or more weeks. In some embodiments, the subject is a human. In some embodiments, the administration of the rapamycin or rapalog results in prolonged survival of the administered cells, relative to a subject that is not administered rapamycin or a rapalog. In some embodiments, the administration of the rapamycin or rapalog increases the frequency of cells circulating in the peripheral blood of a subject, relative to a subject that is not administered rapamycin or a rapalog.

In general, an appropriate dosage and treatment regimen provides the cells, vectors, nucleic acids, or lipid nanoparticles in an amount sufficient to provide therapeutic and/or prophylactic benefit. Such a response can be monitored by establishing an improved clinical outcome (e.g., more frequent remissions, complete or partial, or longer disease-free survival) in treated subjects as compared to non-treated subjects. Decreases (e.g., reductions having statistical significance when compared to a relevant control) in preexisting immune responses to an antigen associated with an autoimmune, allergic, or other inflammatory condition as provided herein generally correlate with an improved clinical outcome. Such immune responses may generally be evaluated using standard leukocyte and/or lymphocyte cell surface marker or cytokine expression, proliferation, cytotoxicity or released cytokine assays, which are routine in the art and may be performed using samples obtained from a subject before and after therapy.

In some embodiments, the subject is a human. In some embodiments, the subject is an animal. In some embodiments, the animal is a research animal. In some embodiments, the animal is a domesticated animal. In some embodiments, the animal is a rodent. In some embodiments, the rodent is a mouse, rat, guinea pig, chinchilla, or hamster. In some embodiments, the animal is a dog, cat, rabbit, guinea pig, hamster, or ferret. In some embodiments, the animal is a bovine, swine, llama, alpaca, sheep, or goat.

### EXAMPLES

### Example 1: Assessment of Activity of Cellular Signaling Controlled by Chimeric Signaling Receptors

FIGS. 9A-9H provides non-limiting examples of chimeric signaling receptors as provided herein.

Chimeric signaling receptors comprising first and second polypeptide combinations were expressed in cells and assessed for activity. Cells expressing chimeric signaling receptors having [mm]CISC2RsL FRB and [mm]CISC2ReL FKBP polypeptides, RISE10RhL FRB and RISE10RhL FKBP polypeptides, and RISE33RhL FRB and RISE33RhL FKBP polypeptides, showed signaling activity after being treated with rapamycin.

### Example 2: Assessment of In Vitro Activation of STAT5 with CISC2 Constructs

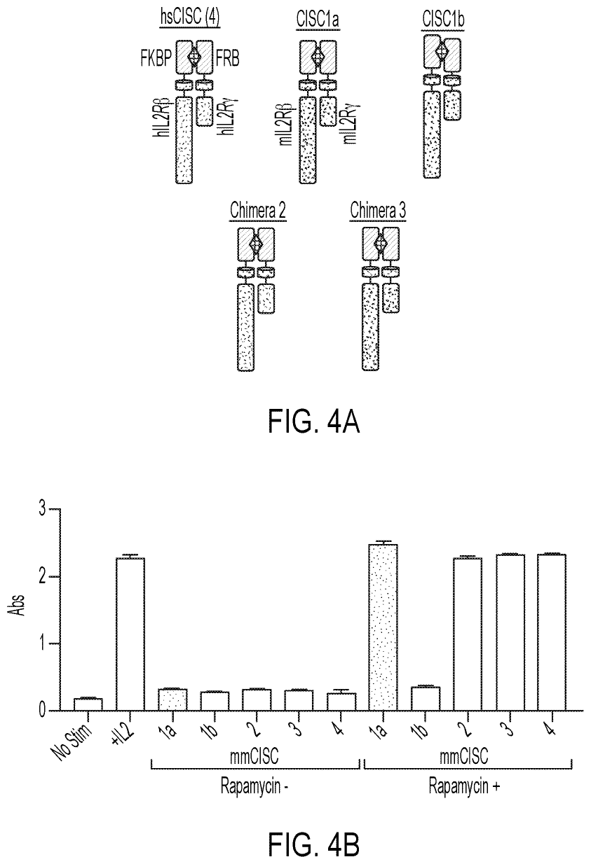

The signaling activity of chimeric signaling receptors illustrated in FIG. 4A was examined. InvivoGen HEK-BLUE™ IL2 reporter cells, in which production of STAT5 leads to release of secreted embryonic alkaline phosphatase (SEAP), were used. The reporter cells were transfected with the chimeric signaling receptors shown in FIG. 4A. After incubation, the reporter cells were exposed to no stimulation (negative control), IL2 (positive control), IL2, or IL2 and rapamycin. A colorimetric enzyme assay was performed to quantify the secreted embryonic alkaline phosphatase, and the results are shown in FIG. 4B. Rapamycin induced the activation of the IL2/STAT5 pathway in all the constructs comprising an extracellular FKBP and FRB domains (CISC1a, Chimera 2, Chimera 3, and hsCISC).

### Example 3: Dose Assessment of In Vitro Activation of ST2 Pathway with RISE33 Constructs

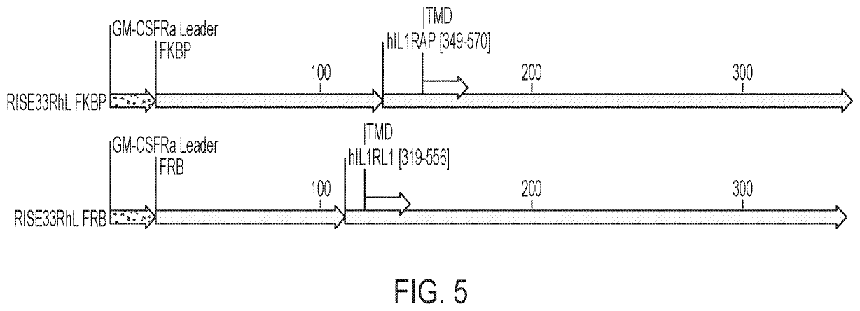

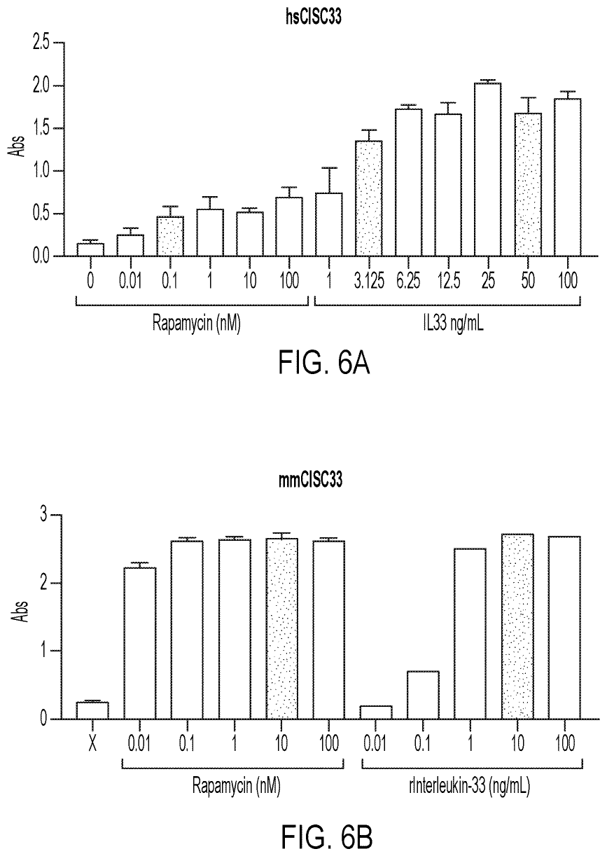

The signaling activity of RISE33 constructs was examined. InvivoGen HEK-BLUE™ IL2 reporter cells, in which production of AP1 and NF-kB leads to release of secreted embryonic alkaline phosphatase (SEAP), were used. The reporter cells were transfected with RISE33 constructs (human and mouse) shown in FIGS. 2 and 5. After incubation, the reporter cells were exposed to different levels of rapamycin or IL33. A colorimetric enzyme assay was performed to quantify the secreted embryonic alkaline phosphatase, and the results are shown in FIG. 6A (human) and FIG. 6B (mouse). CISC33 was activated by stimulation and dose responses to the stimulations were observed.

### Example 4: Assessment of In Vitro Activation of IL10 with RISE10 Constructs

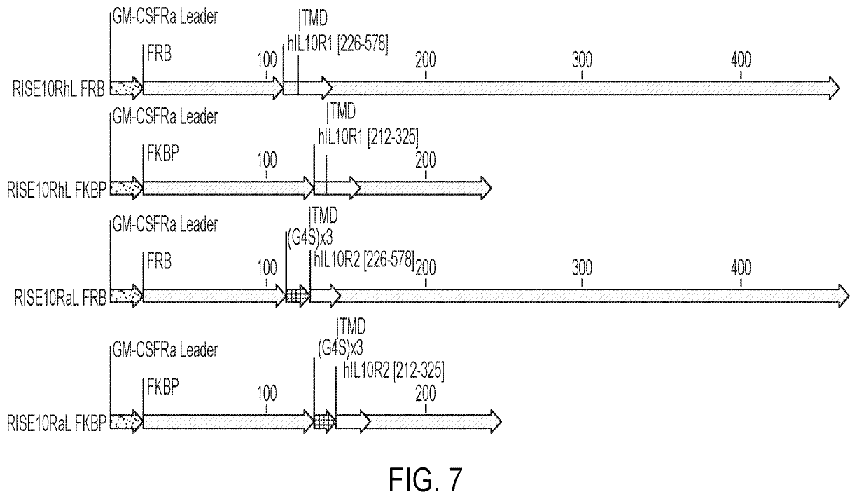

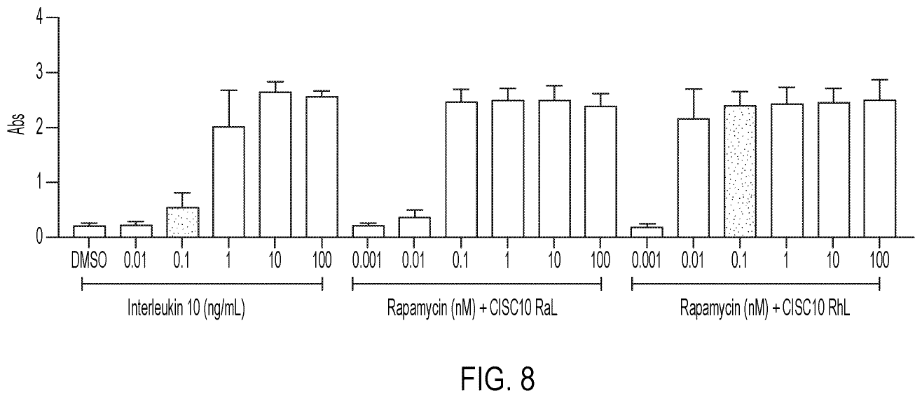

The signaling activity of RISE10 constructs (FIGS. 2 and 7) was examined. InvivoGen HEK-BLUE™ IL2 reporter cells, in which production of STAT3 (from IL10 signaling) leads to release of secreted embryonic alkaline phosphatase (SEAP), were used. The reporter cells were transfected with different levels of IL10, rapamycin and the RISE10 construct with a synthetic linker, (G4S)×3 (“CISC10 RaL”), or rapamycin and the RISE10 construct with IL10 hinge domains (“CISC10 RhL”). A colorimetric enzyme assay was performed to quantify the secreted embryonic alkaline phosphatase, and the results are shown in FIG. 8. The CISC10 RaL cells were observed to reach a peak plateau at a dose of 0.1 nM, while the CISC10 RhL cells were observed to reach peak plateau at a dose of 0.01 nM, which is similar to the IL10 dose of 10 ng/ml. These results indicate that stimulation with rapamycin activates the IL10 signaling pathway through CISC10, and that the CISC10 RhL cells are more sensitive to rapamycin activation than the CISC10 RaL cells at certain lower doses.

### Example 5: Assessment of in vitro Expression of RISE33 Constructs and Related Activation

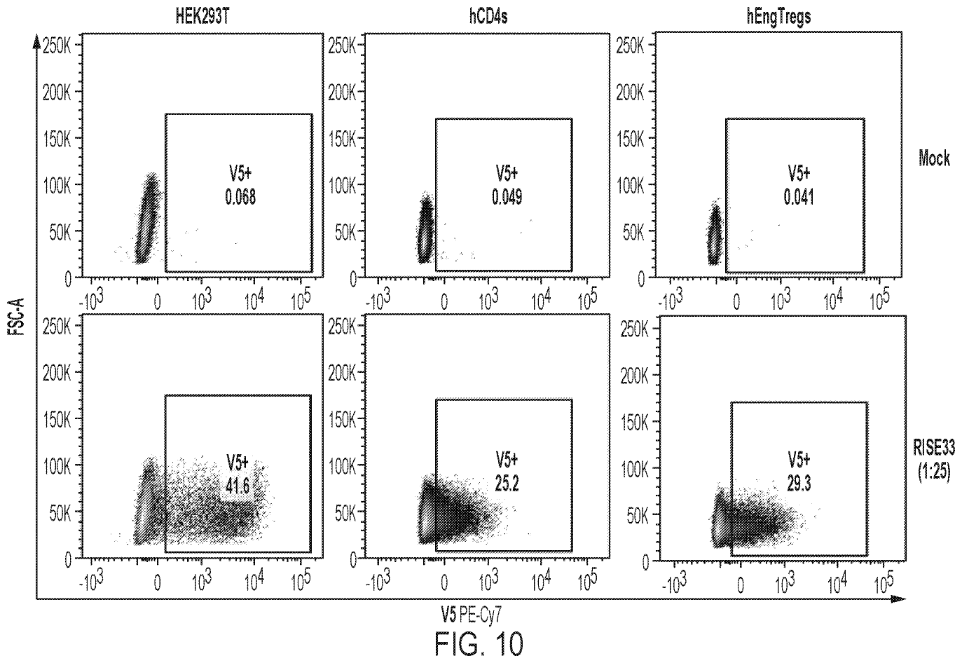

The in vitro expression of RISE33 in human CD4 T cells was examined. Human CD4 T cells were transfected with RISE33 constructs comprising a V5 tag (for flow cytometry). Briefly, the human CD4 T cells were thawed and stimulated with beads (3:1 ratio) (day 0). One day later, 2×10⁵ cells were transduced with lentiviral vectors comprising the RISE33 constructs (day 1) The next day, the cells were supplemented with IL-2 (day 2). The cells were de-beaded and expanded 24 hours later (day 3). On day 6, the cells were phenotyped. The results are shown in FIG. 10 and demonstrate that the RISE33 construct was successfully expressed in T cells.

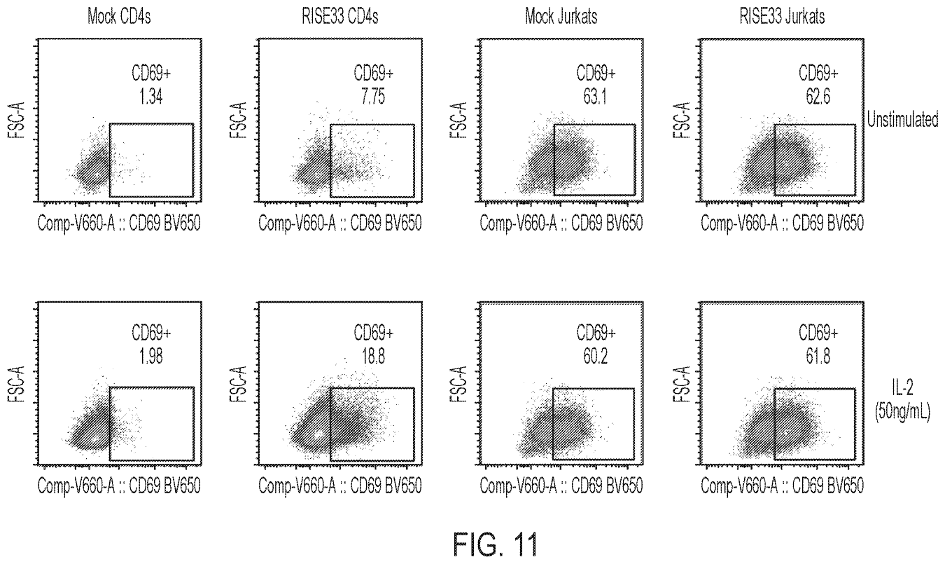

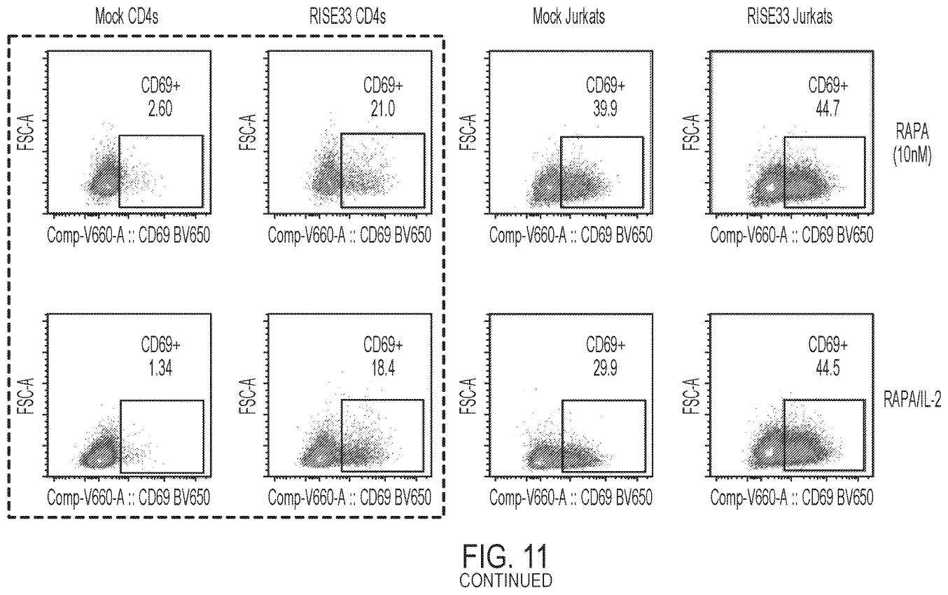

In a further experiment, T cell activation was examined in CD4 T cells and Jurkat cells transfected with the RISE33 construct (controls were mock-transfected T CD4 T cells and mock-transfected Jurkat cells). In this experiment, cells were stained for CD69, which is upregulated after IL-33 signaling (Thierry et al., 2014; Polo et al., 2019). As can be seen from FIG. 11, RISE33-transduced cells had greater T cell activation in the presence of rapamycin. In addition, in cells stimulated with rapamycin (100 nM) for 10 minutes, increased P65-NFκB signaling was observed (0.49% of mock cells vs. 1.32% of RISE33-transduced cells; data not shown), indicating increased activation of the RISE33-transduced T cells in response to rapamycin.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. A chimeric signaling receptor for controlling ST2 signaling in a cell, the chimeric signaling receptor comprising a first polypeptide and a second polypeptide, wherein:
the first polypeptide comprises a first extracellular domain, a first transmembrane domain, and a first membrane proximal signaling domain,
the second polypeptide comprises a second extracellular domain, a second transmembrane domain, and a second membrane proximal signaling domain,
the first extracellular domain comprises the rapamycin binding domain of FK-binding protein 12 (FKBP) or a functional fragment thereof,
the second extracellular domain comprises the rapamycin binding domain of FKBP12-Rapamycin Binding domain of mTOR (FRB) or functional fragment thereof; and wherein
(i) the first membrane proximal signaling domain comprises an intracellular signaling domain or functional fragment of ST2, and the second membrane proximal signaling domain comprises an intracellular signaling domain or functional fragment of IL1RAP; or
(ii) the first membrane proximal signaling domain comprises an intracellular signaling domain or functional fragment of IL1RAP, and the second membrane proximal signaling domain comprises an intracellular signaling domain or functional fragment of ST2.

2. A chimeric signaling receptor for controlling IL18R signaling in a cell, the chimeric signaling receptor comprising a first polypeptide and a second polypeptide, wherein:
the first polypeptide comprises a first extracellular domain, a first transmembrane domain, and a first membrane proximal signaling domain,
the second polypeptide comprises a second extracellular domain, a second transmembrane domain, and a second membrane proximal signaling domain,
the first extracellular domain comprises the rapamycin binding domain of FK-binding protein 12 (FKBP) or a functional fragment thereof,
the second extracellular domain comprises the rapamycin binding domain of FKBP12-Rapamycin Binding domain of mTOR (FRB) or functional fragment thereof; and wherein
(i) the first membrane proximal signaling domain comprises an intracellular signaling domain or functional fragment of IL18R1, and the second membrane proximal signaling domain comprises an intracellular signaling domain or functional fragment of IL18RAP; or
(ii) the first membrane proximal signaling domain comprises an intracellular signaling domain or functional fragment of IL18RAP, and the second membrane proximal signaling domain comprises an intracellular signaling domain or functional fragment of IL18R1.

3. A chimeric signaling receptor for controlling IL36R signaling in a cell, the chimeric signaling receptor comprising a first polypeptide and a second polypeptide, wherein:
the first polypeptide comprises a first extracellular domain, a first transmembrane domain, and a first membrane proximal signaling domain,
the second polypeptide comprises a second extracellular domain, a second transmembrane domain, and a second membrane proximal signaling domain,
the first extracellular domain comprises the rapamycin binding domain of FK-binding protein 12 (FKBP) or a functional fragment thereof,
the second extracellular domain comprises the rapamycin binding domain of FKBP12-Rapamycin Binding domain of mTOR (FRB) or functional fragment thereof; and wherein
(i) the first membrane proximal signaling domain comprises an intracellular signaling domain or functional fragment of IL36R, and the second membrane proximal signaling domain comprises an intracellular signaling domain or functional fragment of IL1RAP; or
(ii) the first membrane proximal signaling domain comprises an intracellular signaling domain or functional fragment of IL1RAP, and the second membrane proximal signaling domain comprises an intracellular signaling domain or functional fragment of IL36R.

4. A chimeric signaling receptor for controlling IL10R signaling in a cell, the chimeric signaling receptor comprising a first polypeptide and a second polypeptide, wherein:
the first polypeptide comprises a first extracellular domain, a first transmembrane domain, and a first membrane proximal signaling domain,
the second polypeptide comprises a second extracellular domain, a second transmembrane domain, and a second membrane proximal signaling domain,
the first extracellular domain comprises the rapamycin binding domain of FK-binding protein 12 (FKBP) or a functional fragment thereof,
the second extracellular domain comprises the rapamycin binding domain of FKBP12-Rapamycin Binding domain of mTOR (FRB) or functional fragment thereof; and wherein
(i) the first membrane proximal signaling domain comprises an intracellular signaling domain or functional fragment of IL10R1, and the second membrane proximal signaling domain comprises an intracellular signaling domain or functional fragment of IL10R2; or
(ii) the first membrane proximal signaling domain comprises an intracellular signaling domain or functional fragment of IL10R2, and the second membrane proximal signaling domain comprises an intracellular signaling domain or functional fragment of IL10R1.

5. A chimeric signaling receptor for controlling LIFR signaling in a cell, the chimeric signaling receptor comprising a first polypeptide and a second polypeptide, wherein:
the first polypeptide comprises a first extracellular domain, a first transmembrane domain, and a first membrane proximal signaling domain,
the second polypeptide comprises a second extracellular domain, a second transmembrane domain, and a second membrane proximal signaling domain,
the first extracellular domain comprises the rapamycin binding domain of FK-binding protein 12 (FKBP) or a functional fragment thereof,
the second extracellular domain comprises the rapamycin binding domain of FKBP12-Rapamycin Binding domain of mTOR (FRB) or functional fragment thereof; and wherein
(i) the first membrane proximal signaling domain comprises an intracellular signaling domain or functional fragment of gp130, and the second membrane proximal signaling domain comprises an intracellular signaling domain or functional fragment of LIFrβ; or
(ii) the first membrane proximal signaling domain comprises an intracellular signaling domain or functional fragment of LIFrβ, and the second membrane proximal signaling domain comprises an intracellular signaling domain or functional fragment of gp130.

6. A chimeric signaling receptor comprising a first polypeptide and a second polypeptide, wherein:
the first polypeptide comprises a first extracellular domain, a first transmembrane domain, a first membrane proximal signaling domain, and a first membrane distal signaling domain, and
the second polypeptide comprises a second extracellular domain, a second transmembrane domain, a second membrane proximal signaling domain, and a second membrane distal signaling domain.

7. The chimeric signaling receptor of claim 6, wherein
the first membrane proximate signaling domain comprises an intracellular signaling domain or functional fragment of ST2,
the second membrane proximate signaling domain comprises an intracellular signaling domain or functional fragment of IL1RAP,
the first membrane distal signaling domain comprises an intracellular signaling domain or functional fragment of IL2Rγ, and
the second membrane distal signaling domain comprises an intracellular signaling domain or functional fragment of IL2Rβ.

8. The chimeric signaling receptor of any one of claims 1-7, further comprising an extracellular linker.

9. The chimeric signaling receptor of claim 8, wherein the extracellular linker comprises (G4S)xN peptide linker or an extracellular hinge of IL1RI, IL1RII, IL1RAP, IL2Rβ, IL2Rγ, IL10R1, IL10R2, IL18R1, IL18RAP, ST2, IL36R, LIFrβ or gp130.

10. A chimeric signaling receptor comprising a first polypeptide and a second polypeptide, wherein the first polypeptide and the second polypeptide each comprise a sequence having at least 80%, at least 85%, at least 90%, at least 95%, at least 96%, at least 97%, at least 98%, at least 99% identity to a sequence selected from SEQ ID NOs: 1-19.

11. The chimeric signaling receptor of claim 10, wherein the first polypeptide and the second polypeptide are each identical to a sequence selected from SEQ ID NOs: 1-19.

12. A nucleic acid encoding the first polypeptide or the second polypeptide of the chimeric signaling receptor of any one of claims 1-11.

13. The nucleic acid of claim 12, wherein the nucleic acid encodes the first polypeptide and the second polypeptide.

14. The nucleic acid of claim 12 or 13, further comprising a promoter that is operably linked to a coding sequence encoding the first polypeptide and/or the second polypeptide.

15. The nucleic acid of claim 14, wherein the promoter is a constitutive promoter.

16. The nucleic acid of claim 15, wherein the constitutive promoter is an EF-1α, a PGK promoter, or an MND promoter.

17. The nucleic acid of claim 16, wherein the promoter is an MND promoter.

18. The nucleic acid of any one of claims 15-17, wherein the promoter is an inducible promoter.

19. The nucleic acid of claim 18, wherein the inducible promoter is inducible by a drug or steroid.

20. A vector comprising the nucleic acid any one of claims 12-19.

21. The vector of claim 20, wherein the vector is a viral vector.

22. The vector of claim 20 or 21, wherein the vector is an adenovirus-associated virus (AAV) vector.

23. The vector of claim 22, wherein the AAV vector is derived from an AAV of serotype AAV1, AAV2, AAV3, AAV4, AAV5, AAV6, AAV7, AAV8, AAV9, AAV10, or AAV11.

24. The vector of claim 21, wherein the viral vector is a lentiviral vector.

25. The vector of claim 20, wherein the vector is a plasmid.

26. The vector of claim 20, wherein the vector is a bacterial artificial chromosome.

27. The vector of claim 20, wherein the vector is a human artificial chromosome.

28. A lipid nanoparticle comprising the nucleic acid of any one of claims 12-19 or the vector of any one of claims 20-27.

29. A cell comprising the chimeric signaling receptor of any one of claims 1-7, or the nucleic acid of any one of claims 12-19.

30. The cell of claim 29, wherein the cell is a stem cell or T cell.

31. The cell of claim 29 or 30, wherein the cell is a CD3+, CD4+, or CD8+ T cell.

32. The cell of any one of claims 29-31, wherein the cell is a Treg cell.

33. The cell of any one of claims 29-32, wherein the cell is a FoxP3+ Treg cell.

34. The cell of any one of claims 29-33, wherein the cell is CTLA-4+, LAG-3+, CD25+, CD39+, CD27+, CD70+, GITR+, neuropilin-1+, galectin-1+, and/or IL-2Rα+.

35. The cell of any one of claims 29-34, wherein the cell promotes Treg expansion.

36. The cell of any one of claims 29-34, wherein the cell has an ST2 phenotype.

37. The cell of any one of claims 29-34, wherein the cell has a Tr1 phenotype.

38. A pharmaceutical composition comprising the cell of any one of claims 29-37 and rapamycin or a rapalog.

39. A method comprising administering to a subject the pharmaceutical composition of claim 38 or the cell of any one of claims 29-37.

40. The method of claim 39, wherein the subject has or is at risk of developing an inflammatory disease, autoimmune disease, allergic disease, or a condition associated with a solid organ transplant.

41. The method of claim 40, wherein the inflammatory disease is selected from pancreatic islet cell transplantation, asthma, hepatitis, traumatic brain injury, primary sclerosing cholangitis, primary biliary cholangitis, polymyositis, stroke, Still's disease, acute respiratory distress syndrome (ARDS), uveitis, inflammatory bowel disease (IBD), ulcerative colitis, graft-versus-host disease (GvHD), tolerance induction for transplantation, transplant rejection, or sepsis.

42. The method of claim 40, wherein the autoimmune disease is type 1 diabetes mellitus, multiple sclerosis, systemic lupus erythematosus, myasthenia gravis, rheumatoid arthritis, early onset rheumatoid arthritis, ankylosing spondylitis, immune-mediated pregnancy loss, immune-mediated recurrent pregnancy loss, dermatomyositis, psoriatic arthritis, Crohn's disease, inflammatory bowel disease (IBD), ulcerative colitis, bullous pemphigoid, pemphigus vulgaris, autoimmune hepatitis, psoriasis, Sjogren's syndrome, or celiac disease.

43. The method of claim 40, wherein the allergic disease is allergic asthma, steroid-resistant asthma, atopic dermatitis, celiac disease, pollen allergy, food allergy, drug hypersensitivity, or contact dermatitis.

44. The method of claim 40, wherein the condition associated with a solid organ transplant is graft-versus-host disease.

45. The method of any one of claims 39-44, wherein the cell is autologous to the subject.

46. The method of any one of claims 39-44, wherein the cell is an allogeneic cell.

47. A method of producing an engineered cell, the method comprising introducing into the cell the nucleic acid of any one of claims 12-19.
